(19) United States
(12) Patent Application Publication
(10) Pub. No.: US 2022/0033405 A1
(43) Pub. Date: Feb. 3, 2022

(54) PHARMACEUTICAL COMPOSITION FOR PREVENTING OR TREATING CANCER CONTAINING PLK1 INHIBITOR AS ACTIVE INGREDIENT

(71) Applicants: NATIONAL CANCER CENTER, Goyang-si (KR); KOREA RESEARCH INSTITUTE OF CHEMICAL TECHNOLOGY, Daejeon (KR)

(72) Inventors: Kyungtae KIM, Seoul (KR); Byung Il LEE, Goyang-si (KR); Joong-Won PARK, Goyang-si (KR); Eun Sook LEE, Gwacheon-si (KR); Sang Jin LEE, Paju-si (KR); Seoung Min BONG, Seoul (KR); Jin Sook KIM, Seoul (KR); Minji PARK, Goyang-si (KR); Eun-Kyung YOON, Goyang-si (KR); Joo-Youn LEE, Daejeon (KR); Su-Hyung LEE, Gunpo-si (KR)

(73) Assignees: NATIONAL CANCER CENTER, Goyang-si (KR); KOREA RESEARCH INSTITUTE OF CHEMICAL TECHNOLOGY, Daejeon (KR)

(21) Appl. No.: 17/297,852

(22) PCT Filed: Nov. 29, 2018
(86) PCT No.: PCT/KR2018/014944

§ 371 Date: May 27, 2021

(30) Foreign Application Priority Data
Nov. 28, 2018 (KR) 10-2018-0149112

(51) Int. Cl. C07D 487/04; A61P 35/00; A23L 33/10

(52) U.S. Cl. CPC C07D 487/04; A23V 2002/00; A23L 33/10; A61P 35/00

(57) ABSTRACT

The present invention relates to a pharmaceutical composition for preventing, treating or alleviating cancer, containing a PLK1 inhibitor as an active ingredient, and a compound according to the present invention selectively binds to PBD of PLK1, thereby having advantages of high selectivity and binding affinity for PLK1 and low toxicity. Therefore, a PLK inhibitor compound according to the present invention can be effectively used as an anticancer agent by inhibiting the growth of various cancer cells, and can be expected to exhibit synergistic effects with existing developed anticancer agents through co-administration, in addition to individual administration thereof.

### TECHNICAL FIELD

The present invention relates to a composition for preventing, alleviating or treating cancer, containing, as an active ingredient, a polo-like kinase 1 (PLK1) inhibitor which inhibits the activity of the protein by binding to the polo-box domain (PBD) of PLK1, and a pharmaceutically acceptable salt thereof.

### BACKGROUND ART

Mitosis refers to a division in which the constituents of all cells are separated into two new cells. When mitosis begins, the condensation of chromosomes, the spindle pole body separation and migration to two poles, the alignment of chromosomes in the middle, and finally the separation of all cellular components occur. When cells begin to divide, chromosomes should form a specific structure for effective bidirectional separation, and such mitotic-specific chromosomal structures usually depend on three multiprotein complexes, two condensin complexes, and a cohesin complex. The cohesin complex binds to its sister chromatids, and the condensin complex serves to make the inside of the chromosome thick and short. Each condensin complex consists of two ATPase subunit heterodimers, a structural maintenance of chromosomes (SMC 2 & SMC 4), and three non-SMC regulatory subunits. A unique set of these three regulatory components will define each condensin complex, and for example, NCAP-D2, NCAP-G, and NCAP-H are constituent elements of condensin complex I, and NCAP-D3, NCAP-G2, and NCAP-H2 are constituent elements of condensin complex II. The SMC 2 and 4 subunit heterodimers are crosslinkers for mitotic DNA condensation using the ATP enzymatic activity thereof. NCAP-H and NCAP-1-12 are kleisin proteins that link the SMC subunit heterodimer and the other two regulatory subunits, and NCAPG NCAPG2, NCAD2, and NCAPD3 are regulatory subunits for each condensin complex containing a HEAT repeat domain corresponding to a variable framework. Condensin complex I is located in cytosol during interphase, is incorporated into the chromosome by aurora kinase B immediately after the collapse of the nuclear membrane, and remains in the chromosome arm until the cytokinesis process. In contrast, condensin complex II causes chromosomes to be condensed during cell division while remaining in the nucleus even in interphase, and condensin complex II is incorporated into the chromosomes by a protein phosphatase 2A (PP2A) catalytic activity-independent function. Various other actions including chromosomal decatenation, chromatin remodeling, and complex I condensation allow chromosome condensation to be maintained until cytokinesis. Further, condensin I present in yeast species is a classical condensin complex for eukaryotic chromosome condensation. Condensin II regulates not only chromosome rigidity, but also various cellular actions such as chromosome segregation, DNA repair, apoptosis, sister chromatid resolution, gene expression regulation, and histone modulation. Interestingly, homozygous mutants of all nematode condensin complex II components exhibit an abnormal size or heterogeneous nuclear distribution. In human cells, a deficiency of any component of condensin complex II results in a defect in chromosome alignment or segregation. In connection with the chromosome segregation action, a recent report has reported that NCAPD3 contributes to the migration of PLK1 to chromosomal cancer.

Chromosome segregation is the most important process for delivering conserved genetic information to each daughter cell. The first step in chromosome segregation is the attachment of microtubule to kinetochore. The kinetochore is a protein complex assembly corresponding to the centromere of the chromosome to which sister chromatids bind. Microtubule-kinetochore binding requires fine regulation by diverse proteins for precise bidirectional interactions. These processes are performed by adjusting the proper time and positioning of such kinase/phosphatase substrate activation through a fine phosphorylation gradient by kinases and phosphatases such as Aurora B and/or PP2A phosphatase.

In such a process, polo-like kinase 1 (PLK1), which is a type of serine/threonine kinase, is known to be essential for chromosome segregation and chromosome integrity. PLK1 mediates the initial stage of the microtubule attachment to the kinetochore. It is located variously in the chromosome, kinetochore, and midbody depending on the migration of microtubules during mitosis, and is located in the kinetochore from prometaphase to metaphase until chromosome alignment in the metaphase plate is completed. Further, when each kinetochore is not properly attached to microtubules, PLK1 located at the kinetochore phosphorylates BubR1 to wait for the start of the anaphase. That is, PLK1 plays a critical role in cell proliferation, acting on various processes in mitosis and DNA damage repair.

Structurally, PLK1 is a type of phosphorylation enzyme, and consists of a kinase site having phosphorylation activity and a Polo-box domain (PBD) that recognizes a substrate, unlike other phosphorylation enzymes. The kinase site and the PBD site form a structure in which phosphorylation enzymatic activity is disturbed when substrates do not compete with each other, and when a substrate binds to the PBD, the kinase site and the PBD site have phosphorylation activity while the structure is opened. Therefore, it is known that most substrates bind to the PBD and are phosphorylated, but when a mutant that suppresses one function of the PBD or KD is created, it seems that the PLK1 function of the cell still remains, so that it is known that even though a substrate binds to the PBD, there are substrates and functions which are irrespective of the KD function. It has been reported that the expression of PLK1, which plays various roles in the process of cell division, is increased in many carcinomas, and in particular, since this expression is fatal to cancer cells, it is known that the inhibition of PLK1 activity induces apoptosis by maintaining a uniaxial spindle fiber state which is abnormal for cells. Therefore, anti-cancer drug development research targeting PLK1 was conducted in various studies. In the initial research stage, PLK1 inhibitors was developed as an ATP competitive inhibitor that suppresses the phosphorylation enzymatic activity of PLK1, and most of the drugs currently in clinical practice as PLK1 inhibitors are such N-terminal ATP binding site inhibitors. However, kinase sites targeted by these inhibitors to suppress phosphorylation activity show similarity with other PLK families or other phosphorylation enzymes, which makes it difficult to selectively target PLK1, and its clinical application is limited due to pharmacodynamic problems even though therapeutic effects are shown in various malignant tumors.

Therefore, the present inventors confirmed from previous studies that NCAPG2, a subunit of condensin complex II, affected PLK1 localization in a kinetochore and substrate phosphorylation activity by binding to the PBD site of PLK1, and by actually investigating the PBD binding site of NCAPG2, a peptide was identified as a PLK1 inhibitor based on this. However, the peptide has limitations such as instability against autolysis and low intracellular permeability.

Therefore, the design of a molecular modeling using a binding structure of the peptide and the PLK1 PBD and the discovery of an effective, low toxicity, and low molecular weight compound which has high binding strength to PLK1 by screening low molecular weight compounds, and as a result, has the ability to inhibit growth of cancer cells have become major challenges, and a study has been conducted on this (Korean Published Patent No. 10-2016-0045957), but studies are still insufficient.

### Disclosure

### Technical Problem

To solve the problems of the present invention as described above, the present inventors screened a library of 340,000 compounds in order to discover a low molecular weight compound having high binding affinity for the PBD of PLK1 and low toxicity by designing a molecular model according to the binding structure of a NCAPG2-derived peptide and the PBD of PLK1, thereby identifying an effective PLK1 inhibiting compound.

In addition, the present inventors found that the growth of various cancer cell lines were efficiently retarded by the compound at the cellular level, thereby completing the present invention based on this.

Thus, an object of the present invention is to provide a composition for preventing, alleviating, or treating cancer, containing a compound represented by the following Chemical Formula 1 or 2, or a pharmaceutically acceptable salt thereof as an active ingredient.

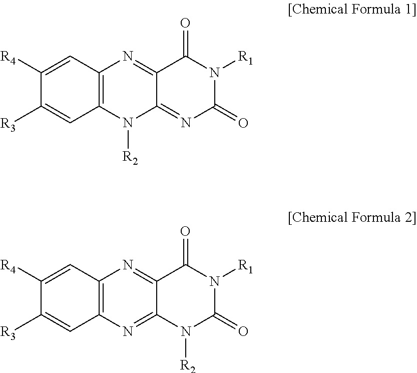

However, technical problems to be solved by the present invention are not limited to the aforementioned problems, and other problems that are not mentioned may be clearly understood by the person skilled in the art from the following description,

### Technical Solution

To achieve the object, the present invention provides a pharmaceutical composition for preventing or treating cancer, containing a compound represented by the following Chemical Formula 1 or 2, or a pharmaceutically acceptable salt thereof as an active ingredient.

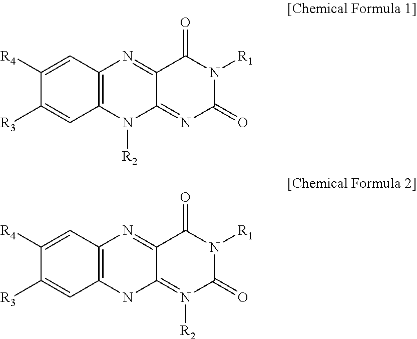

(in Chemical Formula 1 or 2, R₁ is H, an alkyl, or —Cₙ—H₂ₙCOOH (n is an integer from 1 to 4), R₂ is H, an alkyl, —CₘH₂ₘCN, —CₘH₂ₘOR₅, or —CₚH₂ₚ(CH(OH))_{q}R₆, R₅ is a phenyl substituted with one or more C₁₋₃ alkyls, R₆ is H, an alkyl, or —OPH₂O₃, m is an integer from 2 to 4, p is an integer from 1 to 3, and q is an integer from 2 to 4,

R₃ is H, a halogen, —NH₂, an alkyl, or —CH═O, and R₄ is H, an alkyl, —COOH, or —CX₃, and X is a halogen)

Further, the present invention provides a health functional food composition for alleviating cancer, containing a compound represented by the following Chemical Formula 1 or 2, or a pharmaceutically acceptable salt thereof as an active ingredient.

As an exemplary embodiment of the present invention, in Chemical Formula 1 or 2,

R₁ may be H, —CH₃, or —CH₂COOH,

R₂ may be H, —CH₃, —C₂H₄CN, —CH₂ (CH(OH))₃CH₂OH, CH₂(CH(OH))₃OPH₂O₃, or

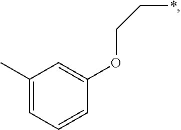

R₃ may be H, Cl, —NH₂, —CH₃, or —CH═O, and

R₄ may be H, —CH₃, —COOH, or —CF₃.

As another exemplary embodiment of the present invention, the compound represented by Chemical Formula 1 or 2 may be selected from the group consisting of the following compounds.
2,4-dioxo-1,2,3,4-tetrahydrobenzo[g]pteridine-7-carboxylic acid;
10-methyl-2H,3H,4H,10H-benzo[g]pteridine-2,4-dione;
8-chloro-1H,2H,3H,4H-benzo[g]pteridine-2,4-dione;
10-methyl-7-(trifluoromethyl)-2H,3H,4H, 1 OH-benzo[g]pteridine-2,4-dione;
8-amino-1,3-dimethyl-1H,2H,3H,4H-benzo[g]pteridine-2,4-dione;
8-amino-2H,3H,4H,10H-benzo[g]pteridine-2,4-dione;
7,8,10-trimethyl-2H,3H,4H,10H-benzo[g]pteridine-2,4-dione;
7,10-dimethyl-2,4-dioxo-2H,3H,4H,10H-benzo[g]pteridine-8-carbaldehyde;
4,10-dihydro-7,8,10-tri methyl-2,4-di oxobenzo[g]pteridine-3 (2H)-acetic acid;
3-{7,8-dimethyl-2,4-dioxo-2H,3H,4H,10H-benzo[g]pteridin-10-yl}propanenitrile;
10-[2-(3-methylphenoxy)ethyl]-7-(trifluoromethyl)-2H,3H,4H,10H-benzo[g]pteridine-2,4-dione;
7,8-dimethyl-10-[(2S,3S,4R)-2,3,4,5-tetrahydroxypentyl]benzo[g]pteridine-2,4-dione; and
[(2R,3S,4S)-5-(7,8-dimethyl-2,4-dioxobenzo[g]pteridin-10-yl)-2,3,4-trihydroxypentyl] dihydrogen phosphate

As still another exemplary embodiment of the present invention, the cancer may be one or more selected from the group consisting of liver cancer, breast cancer, hematologic cancer, cervical cancer, and prostate cancer.

As yet another exemplary embodiment of the present invention, the compound may bind to a polo-box domain (PBD) of polo-like kinase 1 (PLK1).

As yet another exemplary embodiment of the present invention, the composition may inhibit the growth of cancer cells.

As yet another exemplary embodiment of the present invention, the composition may induce apoptosis of cancer cells.

In addition, the present invention provides a method for preventing or treating cancer, the method including: administering a pharmaceutical composition including the compound represented by Chemical Formula 1 or 2, or a pharmaceutically acceptable salt thereof as an active ingredient to an individual.

Furthermore, the present invention provides a use of a pharmaceutical composition comprising the compound represented by Chemical Formula 1 or 2, or a pharmaceutically acceptable salt thereof as an active ingredient for preventing or treating cancer.

### Advantageous Effects

As a result of performing a library screening of compounds to discover a low molecular weight compound having low toxicity while having high binding affinity for the PBD of PLK1 the present inventors identified an effective compound represented by Chemical Formula 1 or 2 of the present invention, and confirmed that the compound effectively bound to the PBD of PLK1 at a low concentration, and remarkably inhibited the growth of liver cancer, breast cancer, hematologic cancer, cervical cancer, and prostate cancer cells.

Thus, the compounds according to the present invention have advantages of having high selectivity and binding affinity for PLK1 and low toxicity by selectively binding to the PBD of PLK1 compared to ATP binding site inhibitors targeting a kinase domain in the related art.

Therefore, a PLK1 inhibitor compound according to the present invention can be effectively used as an anticancer agent by inhibiting the growth of various cancer cells, and can be expected to exhibit synergistic effects with existing developed anticancer agents through co-administration, in addition to individual administration thereof.

### DESCRIPTION OF DRAWINGS

FIG. 1 illustrates the principle of an FP analysis method (fluorescence polarization competition assay) used to discover a low molecular weight compound according to an exemplary embodiment of the present invention that selectively binds to the PBD of PLK1 to suppress the activity of PLK1.

FIG. 2 is a set of graphs showing the FP assay analysis results and IC₅₀ of compounds according to an exemplary embodiment of the present invention.

FIG. 3 illustrates graphs showing FP assay results and IC₅₀ of compounds according to an exemplary embodiment of the present invention.

FIG. 4 illustrates graphs showing FP assay results and IC₅₀ of compounds according to an exemplary embodiment of the present invention.

FIGS. 5A and 5B are graphs for measuring the ability of Compound 2 (M2) to inhibit the growth of cancer cells according to Example 3 of the present invention.

FIG. 5C is a set of graphs for measuring the abilities of M2 and M3 variants to inhibit the growth of cancer cells in JIMT1 cells according to Example 3 of the present invention.

FIG. 6 is a set of graphs for measuring the abilities of Compound 2 (M2), Compound 3 (M4), Compound 4 (M21), and sorafenib to inhibit the growth of liver cancer cell lines according to Example 3 of the present invention.

FIG. 7 is a set of graphs for measuring the ability of Compound 3 (M4) to inhibit the growth of cancer cells according to Example 3 of the present invention.

FIG. 8 is a set of graphs for measuring the ability of Compound 3 (M4) to inhibit the growth of cancer cells according to Example 3 of the present invention.

FIG. 9A is a set of graphs for measuring the abilities of Compound 5 (M23) and Compound 6 (M25) to inhibit the growth of liver cancer cell lines according to Example 3 of the present invention.

FIG. 9B is a set of graphs for measuring the abilities of M2 and M3 variants to inhibit the growth of cancer cells in HepG2 cells according to Example 3 of the present invention.

FIG. 9C is a set of graphs for measuring the abilities of M2 and M3 variants to inhibit the growth of cancer cells in SNU449 cells according to Example 3 of the present invention.

FIG. 10 is a set of graphs for measuring the ability of Compound 2 (M2) alone and the mixed treatment of Compound 2 (M2) and BI2536 to inhibit the growth of liver cancer cell lines according to Example 3 of the present invention.

FIG. 11 confirms the mutual positional relationship among r-tubulin located in the centrosome, PLK1, and the chromosome (DAPI) during treatment of compounds according to an exemplary embodiment of the present invention according to Example 4 of the present invention.

FIG. 12 is a set of photographs and a graph illustrating the degree of staining of NCAPG2 in the chromosome arm and centrosome according to Example 4 of the present invention.

FIG. 13 is a set of graphs illustrating the effect on the cell cycle in the case of treatment with Compound 2 (M2) according to Example 5 of the present invention.

FIG. 14A illustrates the relative cell area of HepG2 cells after treatment with M2 or B12536.

FIG. 14B illustrates images observed through nuclear staining in HepG2 cells treated with M2 or BI2536.

FIG. 15A illustrates the results of performing Flow cytometry after treating HepG2 cells with M2 and BI2536, respectively.

FIG. 15B is a graph illustrating the results of apoptosis after treating HepG2 cells while increasing the doses of M2 and BI2536, respectively.

FIG. 16 is a set of photographs of removing and histopathologically analyzing the lungs, heart, liver, kidneys, spleen, and skin after intraperitoneally injecting Compound 4 and DMSO into mice, respectively, according to Example 8 of the present invention.

FIG. 17 is a set of graphs respectively illustrating changes in tumor size and mouse body weight according to Example 8 of the present invention.

FIG. 18 is a set of photographs illustrating the appearance of the cancer-producing tissues removed according to Example 8 of the present invention.

FIG. 19 is a photograph illustrating that immunohistochemical staining for PLK1 was performed to compare the expression of PLK1 in the removed tissues according to Example 8 of the present invention, the difference in the expression of PLK1 itself was not remarkable, but the number of cells during the mitotic phase was decreased.

FIG. 20A illustrates the macroscopic morphologies and MRI images of tumors transplanted after treating mice with M2 according to Example 9 of the present invention.

FIG. 20B is a graph illustrating a change in the volume of the transplanted tumors and the tumor growth reducing effect of M2 using the MRI images of FIG. 20A.

FIG. 20C illustrates that the number of cells during the mitotic phase is reduced in the tumor tissues transplanted according to Example 9 of the present invention.

FIG. 20D is a graph illustrating the mitotic index calculated for each treatment group using the histopathological observations shown in FIG. 20C.

FIG. 21A illustrates a change in the size of a tumor transplanted after treating mice with M2 according to Example 9 of the present invention by MRI images.

FIG. 21B illustrates the final weight of a tumor transplanted after treating mice with M2 according to Example 9 of the present invention.

FIG. 21C illustrates a change in the volume of a tumor transplanted after treating mice with M2 according to Example 9 of the present invention.

FIG. 22A illustrates MM images of tumors transplanted into a mouse in a control according to Example 10 of the present invention.

FIG. 22B illustrates MRI images of tumors transplanted into a mouse in a group treated with M2 according to Example 10 of the present invention.

FIG. 22C illustrates MRI images of tumors transplanted into a mouse in a group treated with BI2536 according to Example 10 of the present invention.

FIG. 22D is a set of graphs comparing changes in tumor volume, tumor weight, and body weight in a group treated with M2 or BI2536 according to Example 10 of the present invention.

### MODES OF THE INVENTION

Since the present invention may be modified into various forms and include various exemplary embodiments, specific exemplary embodiments will be illustrated in the drawings and described in detail in the Detailed Description. However, the description is not intended to limit the present invention to the specific exemplary embodiments, and it is to be understood that all changes, equivalents, and substitutions belonging to the spirit and technical scope of the present invention are included in the present invention. When it is determined that the detailed description of the related publicly known art in describing the present invention may obscure the gist of the present invention, the detailed description thereof will be omitted.

The present invention relates to a PLK1 inhibitor and a use thereof, and more specifically, to a low toxicity compound having high binding affinity for the PBD of PLK1 and a composition for preventing, alleviating, or treating cancer, containing the compound as an active ingredient. Hereinafter, the present invention will be described in detail.

Through previous studies, the present inventors have found that the GVLSpTLI peptide centered on phosphorylated threonine located at position 1010 of NCAPG2 binds to the polo-box domain (PBD), which is a substrate binding site of serine/threonine-protein kinase 1 (PLK1), and this binding trigger locating the spindle fiber into chromosome, which is very important for the mitotic phase action of PLK1. However, since there are problems in that limitations such as the instability of a peptide and low intracellular permeability need to be overcome when developing the peptide as an anticancer agent, attempts have been made in the present invention to simulate the PBD binding structure of the peptide and discover a low molecular weight compound capable of competitively binding to the PBD based on a crystal structure for the binding site of the peptide and the PLK1 PBD.

Thus, in an exemplary embodiment of the present invention, 700 candidate compounds were derived by performing a primary screening on a library of 340,000 compounds through an in silico assay, and an effective compound that efficiently inhibits the binding between the peptide and PLK1, that is, a PLK1 inhibitor was discovered by performing an FP analysis method on the compounds (see Examples 1 and 2).

In another exemplary embodiment of the present invention, to investigate whether the compound finally discovered through the exemplary embodiment can actually inhibit the growth of various cancer cell lines, measure the number of cells after treating liver cancer, breast cancer, hematologic cancer, cervical cancer, and prostate cancer cell lines with the compound at the cellular level. It was confirmed that the compound effectively inhibited liver cancer, breast cancer, hematologic cancer, cervical cancer, and prostate cancer cells in proportion to the treatment concentration, and it could be confirmed that the inhibitory effect on the relative growth of normal cells was relatively small (see Example 3).

In still another exemplary embodiment of the present invention, it was confirmed that this compounds act differently from phosphorylation activity as a PLK1 inhibitor involved in the normal cell division process in the cancer cells, and it was confirmed that a PBD targeting Hit material prevented the normal position of PLK1 itself in the cell to make the position of the exact partners thereof inappropriate, thereby exhibiting the effect of suppressing the progress of cell growth at the phases prior to the mitotic phase (see Examples 4 and 5).

In yet another exemplary embodiment of the present invention, it was confirmed that as a result of treating HepG2 cells with M2, the antiproliferative effect of cells appeared (see Example 6).

In yet another exemplary embodiment of the present invention, it was confirmed that as a result of treating HepG2 cells with M2, an apoptosis population was increased (see Example 7).

In yet another exemplary embodiment of the present invention, a toxicity test of the compounds and the ability of the compounds to inhibit cancer growth in a liver cancer xenograft model were confirmed (see Example 8).

In yet another exemplary embodiment of the present invention, the ability of the compounds to inhibit cancer growth in a liver cancer orthotopic xenograft model was confirmed (see Examples 9 and 10).

Through the results, a compound represented by the following Chemical Formula 1 or 2 according to the present invention or a pharmaceutically acceptable salt thereof may be used as a therapeutic agent for various carcinomas, particularly, liver cancer, breast cancer, hematologic cancer, cervical cancer, and prostate cancer.

Thus, the present invention provides a pharmaceutical composition for preventing or treating cancer, containing a compound represented by the following Chemical Formula 1 or 2 or a pharmaceutically acceptable salt thereof as an active ingredient.

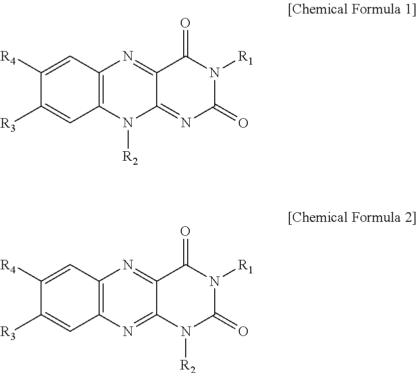

In Chemical Formula 1 or 2,

R₁ is H, an alkyl, or —CₙH₂ₙCOOH (n is an integer from 1 to 4),

R₂ is H, an alkyl, —CₘH₂ₘCN, —CₘH₂ₘOR₅, or —CₚH₂ₚ(CH(OH))_{q}R₆, R₅ is a phenyl substituted with one or more C₁₋₃ alkyls, R₆ is H, an alkyl, or —OPH₂O₃, in is an integer from 2 to 4, p is an integer from 1 to 3, and q is an integer from 2 to 4,

R₃ is II, a halogen, —NH₂, an alkyl, or —CH═O, and

R₄ is II, an alkyl, —COOK, or —CX₃, and X is a halogen.

Preferably, in Chemical Formula 1 or 2,

R₁ may be H, —CH₃, or —CH₂COOH,

R₂ may be H, —CH₃, —C₂H₄CN, —C₂H₄CN, —CH₂(CH(OH))₃CH₂OH, —CH₂ (CH(OH))₃OPH₂O₃, or

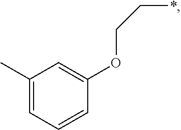

R₃ may be H, Cl, —NH₂, —CH₃, or —CH═O, and

R₄ may be H, —CH₃, —COOH, or —CF₃.

Further, more preferably, the compound represented by Chemical Formula 1 or 2 may be selected from the group consisting of the following compounds.
2,4-Dioxo-1,2,3,4-tetrahydrobenzo[g]pteridine-7-carboxylic acid;
10-methyl-2H,3H,4H, 1 OH-benzo[g]pteridine-2,4-dione,
8-chloro-1H,2H,3H,4H-benzo[g]pteridine-2,4-dione;
10-methyl-7-(trifluoromethyl)-2H, 3H, 4H, 1 OH-benzo[g]pteridine-2,4-dione;
8-amino-1,3-dimethyl-1H,2H,3H,4H-benzo[g]pteridine-2,4-dione;
8-amino-2H,3H,4H,10H-benzo[g]pteridine-2,4-dione;
7,8,10-trimethyl-2H,3H,4H,10H-benzo[g]pteridine-2,4-dione;
7,10-dimethyl-2,4-dioxo-2H, 3H,4H, 1 OH-benzo[g]pteridine-8-carbaldehyde;
4,10-Dihydro-7,8,10-trimethyl-2,4-dioxobenzo[g]pteridine-3(2H)-acetic acid;
3-{7,8-dimethyl-2,4-dioxo-2H,3H,4H, 1 OH-benzo[g]pteridin-10-yl}propanenitrile;
10-[2-(3-methylphenoxy)ethyl]-7-(trifluoromethyl)-2H,3H,4H,10H-benzo[g]pteridine-2,4-dione;
7,8-Dimethyl-10-[(2S,3S,4R)-2,3,4,5-tetrahydroxypentyl]benzo[g]pteridine-2,4-dione; and
[(2R,3S,4S)-5-(7,8-dimethyl-2,4-dioxobenzo[g]pteridin-10-yl)-2,3,4-trihydroxypentyl] dihydrogen phosphate

Hereinafter, 2,4-Dioxo-1,2,3,4-tetrahydrobenzo[g]pteridine-7-carboxylic acid, and derivatives thereof, which are compounds discovered according to Examples 1 and 2 of the present invention will be summarized.

TABLE 1

| NO. | IUPAC NAME | Structural formula |
|---|---|---|
| Compound 1 | 2,4-Dioxo-1,2,3,4- tetrahydrobenzo[g]pteridine-7-carboxylic acid | 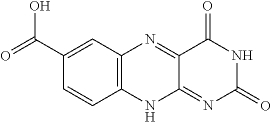 [SMILES: O=C(O)c1ccc2[nH]c3nc(=O)[nH]c(=O)c-3nc2c1] |
| Compound 2 (M2) | 10-methyl-2H,3H,4H,10H- benzo[g]pteridine-2,4-dione | 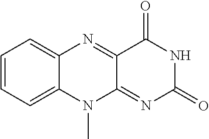 [SMILES: Cn1c2nc(=O)[nH]c(=O)c-2nc2ccccc21] |
| Compound 3 (M4) | 8-chloro-1H,2H,3H,4H- benzo[g]pteridine-2,4-dione | 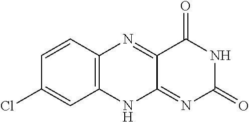 [SMILES: O=c1nc2[nH]c3cc(Cl)ccc3nc-2c(=O)[nH]1] |
| Compound 4 (M21) | 10-methyl-7-(trifluoromethyl)- 2H,3H,4H,10H-benzo[g]pteridine-2,4- dione | 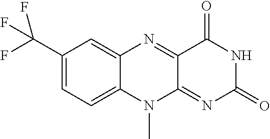 [SMILES: Cn1c2nc(=O)[nH]c(=O)c-2nc2cc(C(F)(F)F)ccc21] |
| Compound 5 (M23) | 8-amino-1,3-dimethyl-1H,2H,3H,4H- benzo[g]pteridine-2,4-dione | 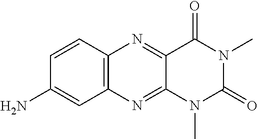 [SMILES: Cn1c(=O)c2nc3ccc(N)cc3nc2n(C)c1=O] |
| Compound 6 (M25) | 8-amino-2H,3H,4H,10H- benzo[g]pteridine-2,4-dione | 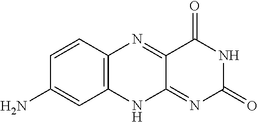 [SMILES: Nc1ccc2nc3c(=O)[nH]c(=O)nc-3[nH]c2c1] |
| Compound 7 (M202) | 7,8,10-trimethyl-2H,3H,4H,10H- benzo[g]pteridine-2,4-dione | 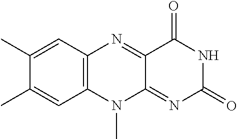 [SMILES: Cc1cc2nc3c(=O)[nH]c(=O)nc-3n(C)c2cc1C] |
| Compound 8 (M203) | 7,10-dimethyl-2,4-dioxo-2H,3H,4H,10H- benzo[g]pteridine-8-carbaldehyde | 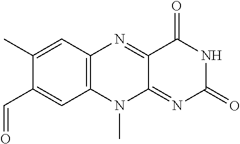 [SMILES: Cc1cc2nc3c(=O)[nH]c(=O)nc-3n(C)c2cc1C=O] |
| Compound 9 (M204) | 4,10-dihydro-7,8,10-trimethyl-2,4- dioxobenzo[g]pteridine-3(2H)-acetic acid | 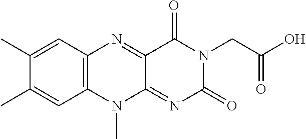 [SMILES: Cc1cc2nc3c(=O)n(CC(=O)O)c(=O)nc-3n(C)c2cc1C] |
| Compound 10 (M206) | 3-{7,8-dimethyl-2,4-dioxo- 2H,3H,4H,10H-benzo[g]pteridin-10- yl}propanenitrile | 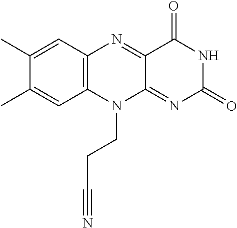 [SMILES: Cc1cc2nc3c(=O)[nH]c(=O)nc-3n(CCC#N)c2cc1C] |
| Compound 11 (M209) | 10-[2-(3-methylphenoxy)ethyl]-7- (trifluoromethyl)-2H,3H,4H,10H- benzo[g]pteridine-2,4-dione | 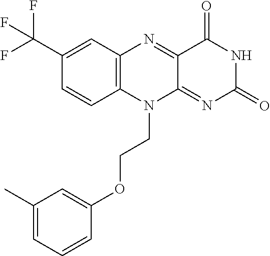 [SMILES: Cc1cccc(OCCn2c3nc(=O)[nH]c(=O)c-3nc3cc(C(F)(F)F)ccc32)c1] |
| Compound 12 (M217) | 7,8-Dimethyl-10-[(2S,3S,4R)-2,3,4,5- tetrahydroxypentyl]benzo[g]pteridine-2,4- dione | 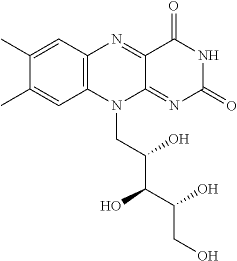 [SMILES: Cc1cc2nc3c(=O)[nH]c(=O)nc-3n(C[C@H](O)[C@H](O)[C@H](O)CO)c2cc1C] |
| Compound 13 (M218) | [(2R,3S,4S)-5-(7,8-dimethyl-2,4- dioxobenzo[g]pteridin-10-yl)-2,3,4- trihydroxypentyl]dihydrogen phosphate | 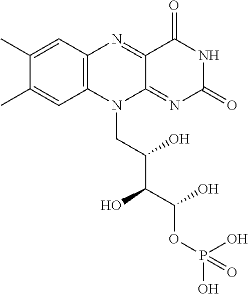 [SMILES: Cc1cc2nc3c(=O)[nH]c(=O)nc-3n(C[C@H](O)[C@H](O)[C@H](O)OP(=O)(O)O)c2cc1C] |

“Cancer”, which is a disease to be prevented or treated by the pharmaceutical composition of the present invention, collectively refers to diseases caused by cells having aggressive characteristics in which the cells ignore normal growth limits and divide and grow, invasive characteristics of infiltrating surrounding tissues, and metastatic characteristics of spreading to other sites in the body. In the present invention, the cancer may be one or more selected from the group consisting of liver cancer, breast cancer, hematologic cancer, prostate cancer, ovarian cancer, pancreatic cancer, gastric cancer, colorectal cancer, brain cancer, thyroid cancer, bladder cancer, esophageal cancer, uterine cancer, and lung cancer, and may be more preferably liver cancer, breast cancer, hematologic cancer, cervical cancer, or prostate cancer, but is not limited thereto.

Unless otherwise mentioned, all technical and scientific terms used herein have the same meaning as commonly understood by the person skilled in the art to which the present invention pertains. Therefore, for example, the term “alkyl” refers to a monovalent group, derived from a straight or branched chain saturated hydrocarbon by removal of a single atom. having 1 to 8 carbon atoms, preferably 1 to 6 carbon atoms.

“Halogen” refers to fluorine, chlorine, bromine, and iodine.

As used herein, the term “prevention” refers to all actions that suppress or delay the onset of cancer by administering the pharmaceutical composition according to the present invention.

As used herein, the term “treatment” refers to all actions that ameliorate or beneficially change symptoms caused by cancer by administering the pharmaceutical composition according to the present invention.

In the present invention, an acid addition salt formed by a pharmaceutically acceptable free acid is useful as the salt. The acid addition salt is obtained from inorganic acids such as hydrochloric acid, nitric acid, phosphoric acid, sulfuric acid, hydrobromic acid, hydroiodic acid, nitrous acid or phosphorous acid, and non-toxic organic acids such as aliphatic mono- and dicarboxylates, phenyl-substituted alkanoates, hydroxyalkanoates and alkanedioates, aromatic acids, aliphatic and aromatic sulfonic acids. These pharmaceutically non-toxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitro benzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate, or mandelate.

The acid addition salt according to the present invention may be prepared by typical methods, for example, dissolving a compound represented by Chemical Formula 1 or 2 in an excess aqueous acid solution, and precipitating this salt using a water-miscible organic solvent, for example, methanol, ethanol, acetone or acetonitrile. Further, the acid addition salt may also be prepared by evaporating the solvent or excess acid from this mixture, and then drying the mixture or suction-filtering a precipitated salt.

In addition, a pharmaceutically acceptable metal salt may be prepared using a base. An alkali metal or alkaline earth metal salt is obtained by, for example, dissolving the compound in an excess alkali metal hydroxide or alkaline-earth metal hydroxide solution, filtering the non-soluble compound salt, evaporating the filtrate, and drying the resulting product. In this case, preparing a sodium, potassium or calcium salt as the metal salt is pharmaceutically suitable. A silver salt corresponding to this is obtained by reacting the alkali metal or alkaline earth metal salt with a suitable silver salt (for example, silver nitrate).

The pharmaceutical composition according to the present invention includes the compound represented by Chemical Formula 1 or 2, or a pharmaceutically acceptable salt thereof as an active ingredient, and may also include a pharmaceutically acceptable carrier. The pharmaceutically acceptable carrier is typically used in formulation, and includes saline, sterile water, Ringer's solution, buffered saline, cyclodextrin, a dextrose solution, a maltodextrin solution, glycerol, ethanol, liposome, and the like, but is not limited thereto, and may further include other typical additives such as an antioxidant and a buffer, if necessary. Further, the composition may be formulated into an injectable formulation, such as an aqueous solution, a suspension, and an emulsion, a pill, a capsule, a granule, or a tablet by additionally adding a diluent, a dispersant, a surfactant, a binder, a lubricant, and the like. With regard to suitable pharmaceutically acceptable carriers and formulations, the composition may be preferably formulated according to each ingredient by using the method disclosed in Remington's literature. The pharmaceutical composition of the present invention is not particularly limited in formulation, but may be formulated into an injection, an inhalant, an external preparation for skin, an oral medication, or the like.

The pharmaceutical composition of the present invention may be orally administered or may be parenterally administered (for example, applied intravenously, subcutaneously, and through the skin, the nasal cavity, or the respiratory tract) according to the target method, and the administration dose may vary depending on the patient's condition and body weight, severity of disease, drug form, and administration route and period, but may be appropriately selected by a person skilled in the art.

The composition of the present invention is administered in a pharmaceutically effective amount. In the present invention, “pharmaceutically effective amount” means an amount sufficient to treat diseases at a reasonable benefit/risk ratio applicable to medical treatment, and an effective dosage level may be determined according to factors including type of diseases of patients, the severity of disease, the activity of dnigs, sensitivity to drugs, administration time, administration route, excretion rate, treatment period, and simultaneously used drugs, and other well known factors in the medical field. The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this amount may be easily determined by those skilled in the art.

Specifically, the effective amount of the composition according to the present invention may vary depending on the patient's age, gender, and body weight, and generally, 0.001 to 150 mg of the composition and preferably, 0.01 to 100 mg of the composition, per 1 kg of the body weight, may be administered daily or every other day or may be administered once to three times a day. However, since the effective amount may be increased or decreased depending on the administration route, the severity of obesity, gender, body weight, age, and the like, the dosage is not intended to limit the scope of the present invention in any way.

As another aspect of the present invention, the present invention provides a health functional food composition for alleviating cancer, containing the compound represented by Chemical Formula 1 or 2, or a pharmaceutically acceptable salt thereof as an active ingredient.

The term “alleviation” used in the present invention refers to all actions that at least reduce a parameter associated with a condition to be treated, for example, the degree of symptoms.

The food composition according to the present invention may be used by adding an active ingredient as it is to food or may be used together with other foods or food ingredients, but may be appropriately used by a typical method. The mixing amount of the active ingredient may be suitably determined depending on its purpose of use (for prevention or alleviation). In general, when a food or beverage is prepared, the composition of the present invention is added in an amount of 15 wt % or less, preferably 10 wt % or less based on the raw material. For long-term intake for the purpose of health and hygiene or for the purpose of health control, however, the amount may be below the above-mentioned range.

Other ingredients are not particularly limited, except that the health functional food composition of the present invention contains the active ingredient as an essential ingredient at an indicated ratio, and the food composition of the present invention may contain various flavorants, natural carbohydrates, and the like as an additional ingredient as in a typical beverage. Examples of the above-described natural carbohydrates include typical sugars such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, and erythritol. As the flavorant except for those described above, a natural flavorant (thaumatin, a stevia extract (for example, rebaudioside A, glycyrrhizin and the like), and a synthetic flavorant (saccharin, aspartame and the like) may be advantageously used. The proportion of the natural carbohydrate may be appropriately determined by the choice of a person skilled in the art.

The health functional food composition of the present invention may contain various nutrients, vitamins, minerals (electrolytes), flavoring agents such as synthetic flavoring agents and natural flavoring agents, colorants and fillers (cheese, chocolate, and the like), pectic acid and salts thereof, alginic acid and salts thereof, organic acids, protective colloid thickeners, pH adjusting agents, stabilizers, preservatives, glycerin, alcohols, carbonating agents used in a carbonated beverage, or the like, in addition to the additives. These ingredients may be used either alone or in combinations thereof. The ratio of these additives may also be appropriately selected by a person skilled in the art.

Hereinafter, preferred Examples for helping the understanding of the present invention will be suggested. However, the following Examples are provided only to more easily understand the present invention, and the contents of the present invention are not limited by the following Examples.

### Example 1. Compound Screening Using Fluorescence Polarization (FP) Method

Through previous studies, the present inventors have found that the GVLSpTLI peptide centered on phosphorylated threonine located at position 1010 of NCAPG2 binds to the polo-box domain (PBD) and dissolve the crystal structure for this binding, which is a substrate binding site of serine/threonine-protein kinase 1 (PLK1), and this binding trigger a binding site of the spindle fiber into the chromosome, which is very important for the mitotic phase action of PLK1. Based on these study results, the present inventors simulated the PBD-binding structure of the peptide and attempted to discover a low molecular weight compound capable of competitively binding to the PBD.

Therefore, the Korean Institute of Chemistry conducted a primary screening of a library of 340,000 compounds through an in silico assay, and conducted an experiment on 700 candidate compounds obtained therefrom.

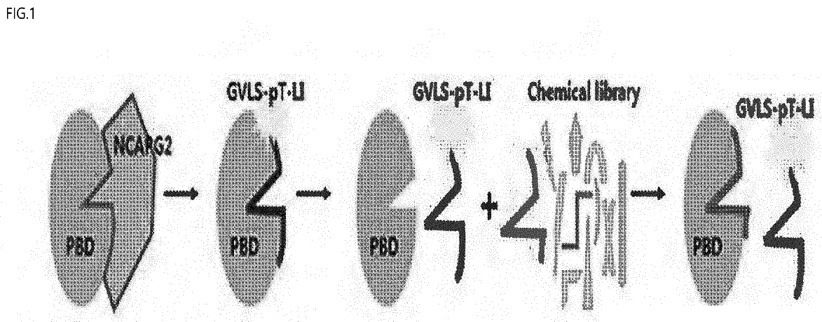

For this purpose, a fluorescence polarization competition assay was performed by mixing a conjugate of a PBD site of PLK1 purified in a solution and a peptide (FITC-labeled 1010pT (GVLSpTLI-NH₂)) to which FITC fluorescence was bound with a low molecular weight compound to be screened. The principle of the analysis method is illustrated in FIG. 1, and is, as illustrated in FIG. 1, a principle of measuring, when a low molecular weight compound capable of competitively binding to the same binding site is bound to the binding site in a state in which a fluorescence-conjugated peptide binds to the PBD domain of PLK1, the degree to which fluorescence is reduced while the peptide is detached from PLK1 to measure the binding strength of the low molecular weight compound to the PLK1.

More specifically, after a reaction was performed at room temperature for 30 minutes by preparing a 4 μM PLK1-PBD protein, a 10 nM peptide (FITC-labeled 1010pT (GVLSpTLI-NH₂)), and a 20 μl candidate compound at each concentration and putting the respective components into a black 96-well plate for mixing, fluorescence polarization (mp) values were measured using Infinite F200 Pro (TECAN Group Ltd, Switzerland). An average value was derived by performing this experiment three times by the same method, and the excitation wavelength and the emission wavelength were set at 485 nm and 535 nm, respectively.

As a result of screening the candidate compounds by the above method, a compound showing a fluorescence polarization value of 180, which is remarkably lower than that measured at about 300 in the case of no compound being added (when only an FITC-labeled 1010pT peptide and a PLK1-PBD protein were added), that is, 2,4-dioxo-1,2,3,4-tetrahydrobenzo pteridine-7-carboxylic acid was discovered, the compound was determined as a hit compound, and the following experiment was performed.

### Example 2. IC₅₀ Measurement of Hit Compound and Derivative Compounds

The IC₅₀ of the compound was intended to be analyzed by performing the FP assay shown in Example 1 on the compound and the hit compound discovered by the primary and secondary compound screening and various derivatives thereof. For this purpose, a target protein to which GST-tag was bound was isolated using a GST resin, and 15 mg/ml of the pure target protein to which GST-tag was bound was obtained by finally performing gel filtration. The target protein was diluted with a reaction buffer and prepared at each of concentrations of 12 uM, 3 uM, and 1.5 uM, and an FITC-bound peptide (FITC-labeled 1010pT (GVLS-pT-LI-NH₂)) stored in a brown tube was diluted with a reaction buffer and prepared at a concentration of 30 nM. Further, the compound at a concentration of 100 mM was diluted with a reaction buffer and prepared at each of 160.0 uM, 80.0 uM, 40.0 uM, 20.0 uM, 10.0 uM, 5.0 uM, 2.5 uM, 1.25 uM, 0.625 uM, 0.3125 uM, 0.15625 uM, and 0.0 uM. Next, the target protein at three concentrations was aliquoted in 12 wells of a 96-well black plate, that is, 12 wells each in 3 rows, and a binding peptide was mixed with the target protein by being aliquoted in each well in which the target protein was aliquoted. Then, the compound at each concentration was aliquoted in each well in which the target protein and the binding peptide were mixed, and reacted at room temperature for 30 minutes. When the reaction was completed, the fluorescence polarization value was measured using Infinite F200 Pro (TECAN Group Ltd, Switzerland) after setting the excitation wavelength and the emission wavelength to 485 nm and 535 nm, respectively, and setting the G-Factor to 1.077. In this case, since the G-Factor slightly differs depending on the characteristics of the peptide, only the peptide was sampled before the start of the experiment to fix the value before use. Binding curves were analyzed using Graphpad Prism (GraphPad Software, San Diego, Calif., USA).

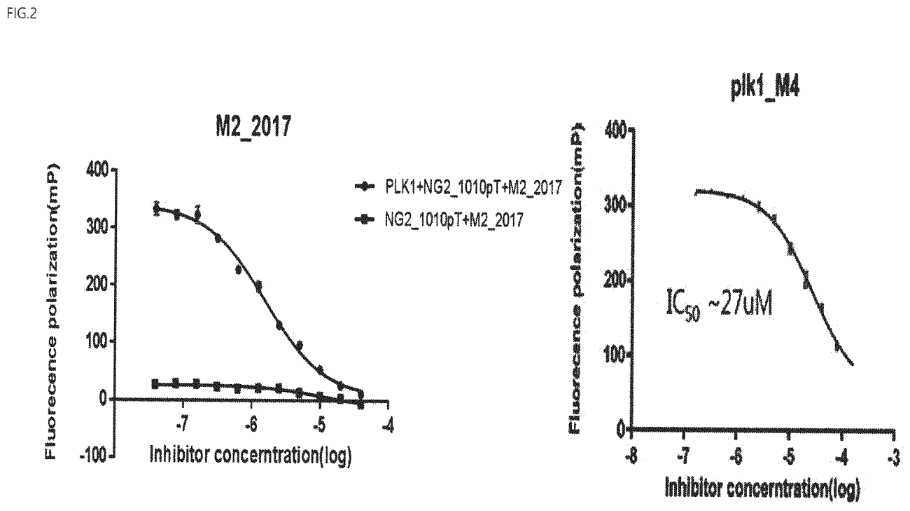

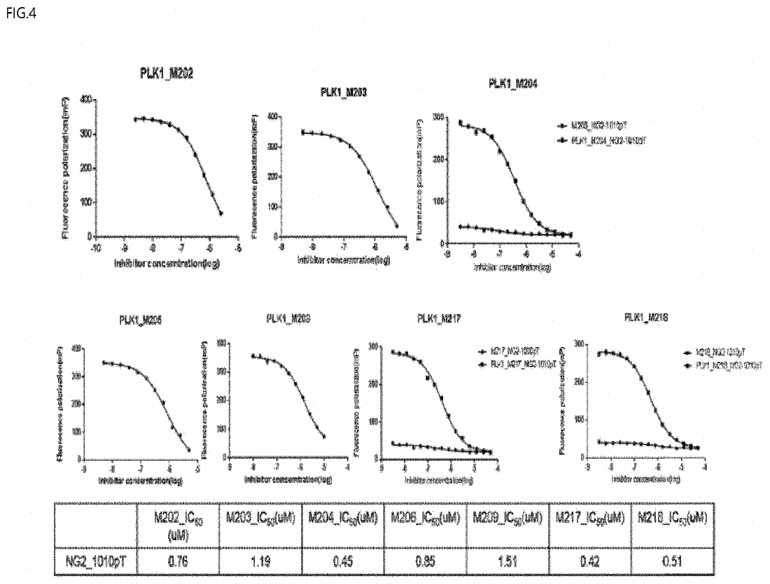

As a result of the experiment, the FP assay analysis results according to the concentration of the compound were obtained and are illustrated in FIGS. 2 to 4, and the IC₅₀ of the compound was calculated based on the results. As a result, the value of 2,4-dioxo-1,2,3,4-tetrahydrobenzo [g] pteridine-7-carboxylic acid was measured to be about 25 μM, and as illustrated in FIGS. 2 to 4, IC₅₀ values of derivatives of the compound were measured to be 0.45 to 27 μM.

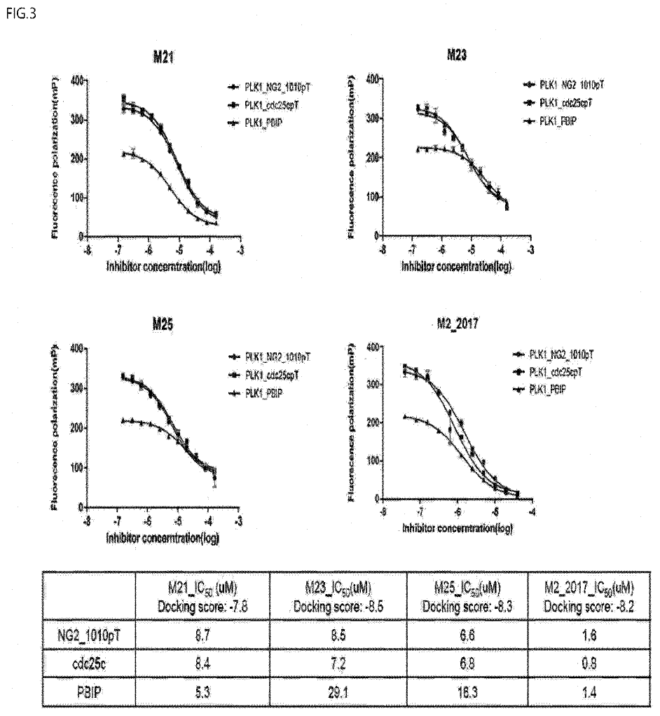

Meanwhile, in the case of Compound 2 (M2), Compound 4 (M21), Compound 5 (M23), and Compound 6 (M25), the IC₅₀ value of FITC-labeled 1010pT (FITC-GVLSpTLI-NH₂), Cdc25cpT (FITC-LLCSpTPN-NH₂), and the PBIP peptide (FITC-LHSpTA-NH₂) were measured, and are illustrated in FIG. 3.

### Example 3. Analysis of Abilities of Hit Compound and Derivative Compounds to Inhibit Growth of Various Cancer Cells

It was intended to investigate whether the compounds that specifically bind to the PBD domain of PLK1 discovered through Examples 1 and 2 actually bind to PLK1 during the division of cancer cells to suppress the division of cells and inhibit the growth of cells.

For this purpose, experiments were performed using liver cancer, breast cancer, hematologic cancer, cervical cancer, and prostate cancer cell lines, a murine liver cancer cell line HEPA 1-6 and a breast cancer cell line MDA-MB-468 were cultured in a DMEM medium supplemented with 10% fetal bovine serum (FBS) and 1% penicillin/streptomycin, the other cell lines were cultured in a RPMI1640 medium supplemented with the same additives, and the cell lines were used in the experiment.

To examine the ability of the compound to inhibit the growth of the breast cancer cell line, the compounds was treated at each indicated μM concentration 1 and 3 days after cell attachment, and the control was treated with a 0.1% solvent (DMSO). After another two days, the cell lines that were attached to the culture plate and grew were rinsed with 1×PBS and treated with 4% paraformaldehyde at room temperature for 10 minutes to fix the cells. Then, after cells were rinsed twice with PBS, the fixed cells were treated with a 0.5% Triton X-100 solution and reacted at room temperature for 15 minutes, rinsed another three times with PBS, and then treated with a DAPI reagent at 0.5 μg/ml, and reacted at 37° C. for 10 minutes to stain the cell nuclei. After the cells were additionally rinsed once with PBS, cells stained with DAPI were photographed by Cytation 3, and the resulting images were analyzed with Gen5 software (Biotek, USA). Meanwhile, cells that grew in a floating manner without being attached to the culture plate were treated with a 4% paraformaldehyde solution, reacted at room temperature for 10 minutes to fix the cells, and then photographed in a bright field by Cytation3, and the resulting images were analyzed by Gen5 software (Biotek, USA).

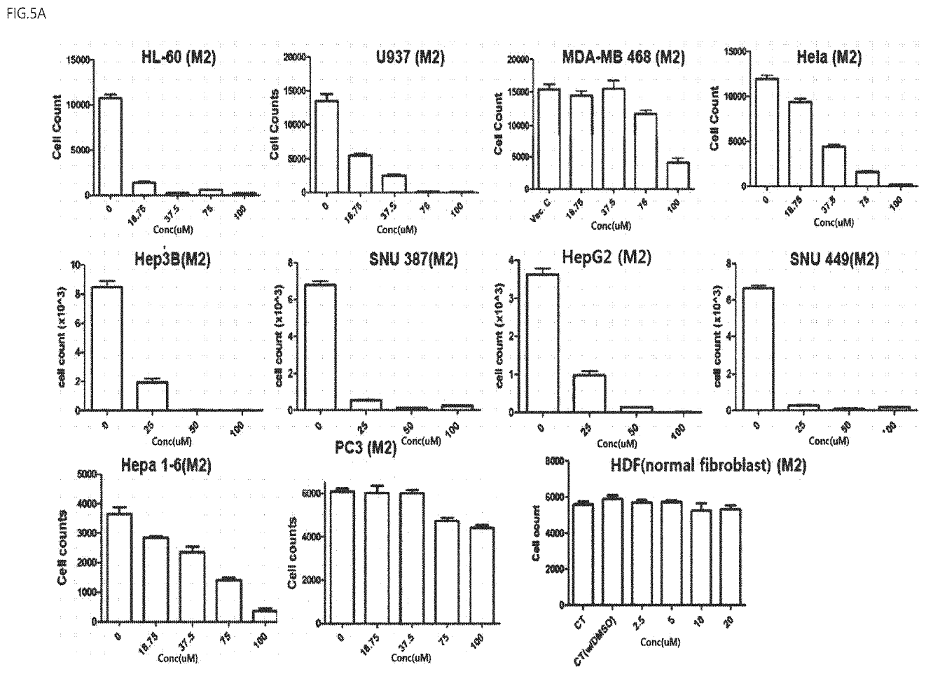

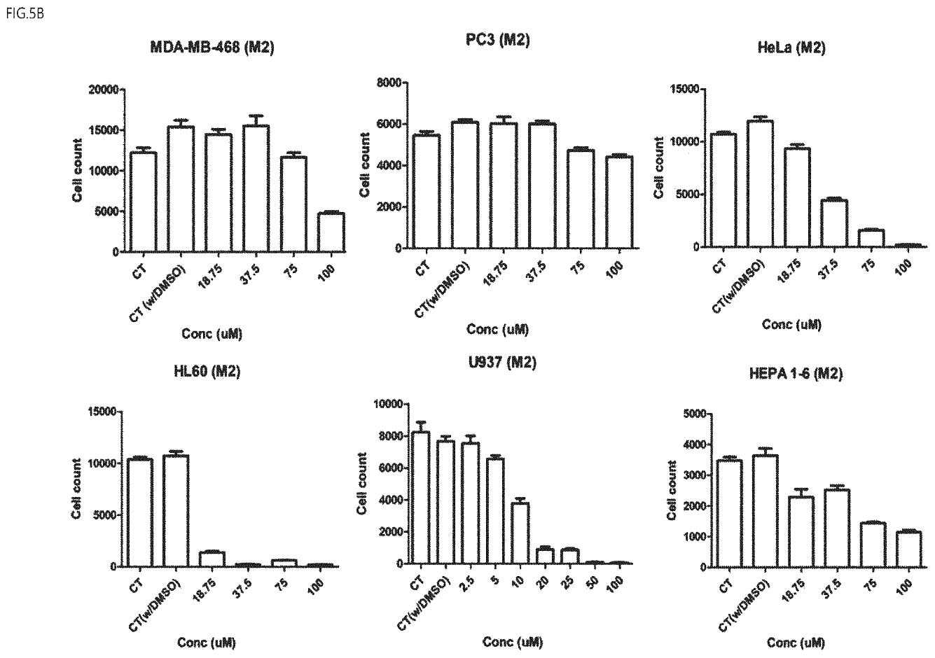

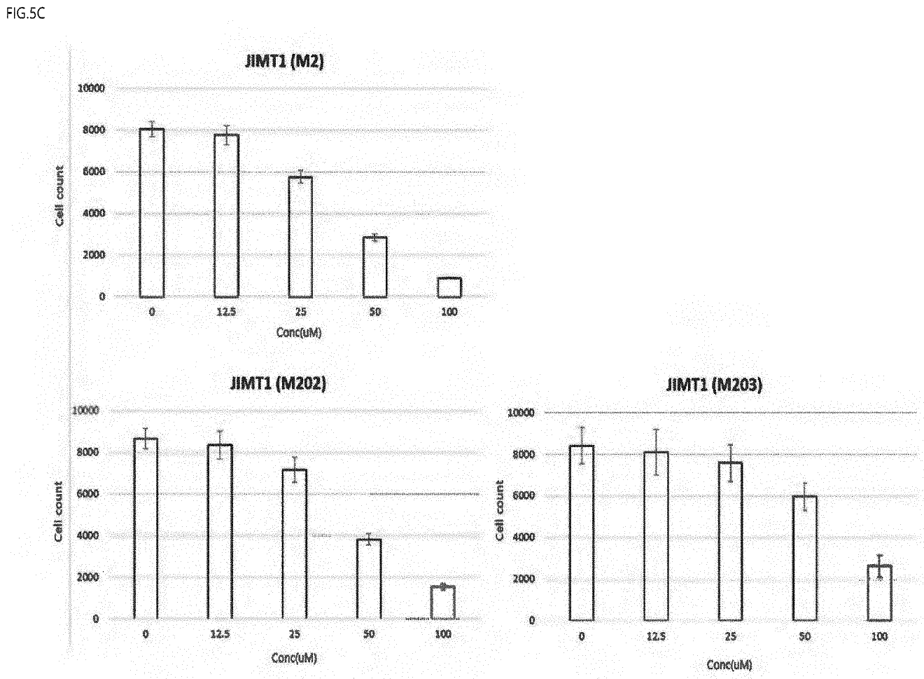

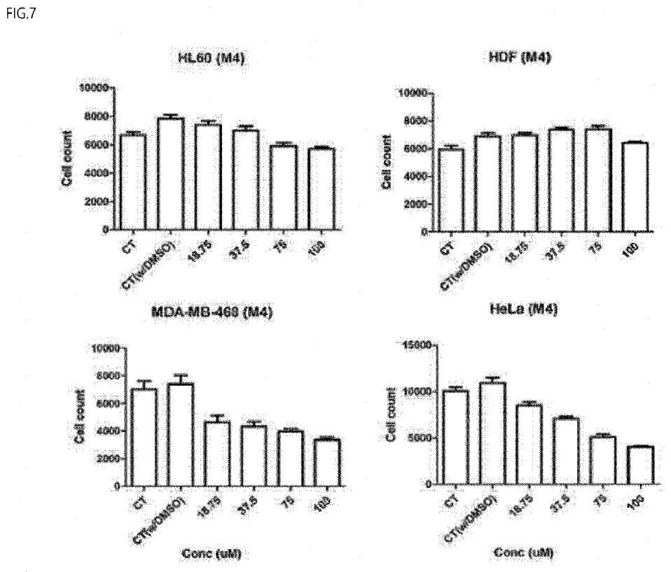

2×10³ MDA-MB-468 cells per well were aliquoted in a 96-well plate, cultured in the same manner as described above, and treated with compound 2 (M2) and compound 3 (M4), and then the ability to inhibit the growth of cells was analyzed. As a result, as illustrated in FIGS. 5A, 5B, and 7, it was confirmed that the number of cells was remarkably reduced in proportion to the treatment concentration of the compounds.

Furthermore, to determine the reactivity of breast cancer cells in M2 variants, 2×10³ cells per well were aliquoted in a 96-well plate using JIMT1 human breast cancer cells and cultured in the same manner as described above, and as a result of additionally performing an experiment, as illustrated in FIG. 5C, it was confirmed that all of M2, M202 and M203 remarkably reduced the number of cancer cells in a dose-dependent manner.

Further, to investigate the ability of Compounds 2 (M2) and 3 (M4) to inhibit the growth of hematologic cancer cell lines, 1×10³ cells per well of hematologic cancer cell lines HL-60 and U937 were aliquoted in 96-well plates, and an experiment was performed in the same manner as described above.

As a result, as illustrated in FIGS. 5A, 5B, and 7, the compounds showed a very high ability to inhibit the growth of cells in both cell lines.

In addition, in order to analyze the ability of Compounds 2 (M2) and 3 (M4) to inhibit the growth in cervical cancer and prostate cancer cell lines, a cervical cancer cell line HeLa and PC-3 cells, which are a prostate cancer cell line, were aliquoted in a 96-well plate and treated with the compounds at various concentrations in the same manner as described above, and then the number of cells was measured.

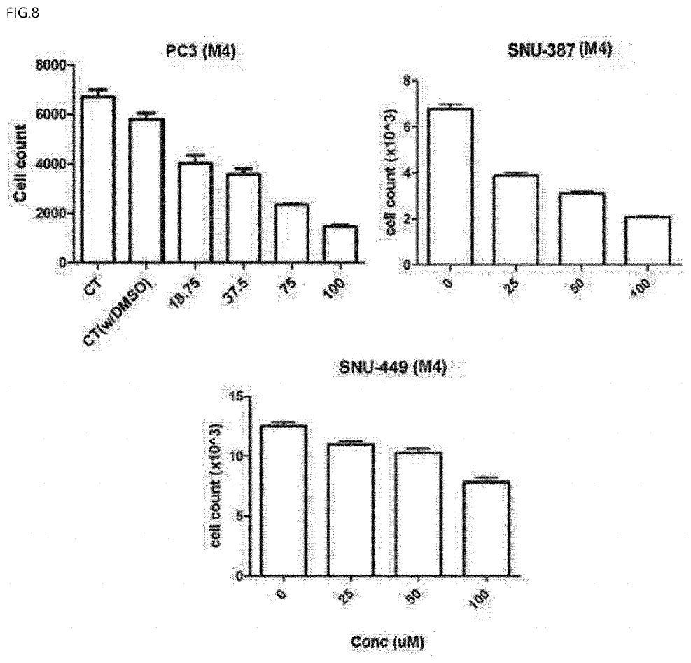

As a result, as illustrated in FIGS. 5A, 5B, and 7, the ability to inhibit the growth of cells according to the treatment with the compounds was confirmed in cervical cancer, and as illustrated in FIGS. 5A, 5B, and 8, it was confirmed that there was a difference in the effect in prostate cancer cells depending on the variants of the compounds.

To analyze the ability to inhibit the growth of liver cancer cell lines, 6.6×10³ HepG2 cells per well, 1×10³ cells of each of Hep3B, SNU-475, and SNU-449 per well, and 2×10³ SNU-387 cells per well were aliquoted in a 96-well plate, cultured in the same manner as described above, treated with Compound 2 (M2), Compound 3 (M4), Compound 4 (M21), Compound 5 (M23), and Compound 6 (M25), and then the ability to inhibit the growth of cells was analyzed.

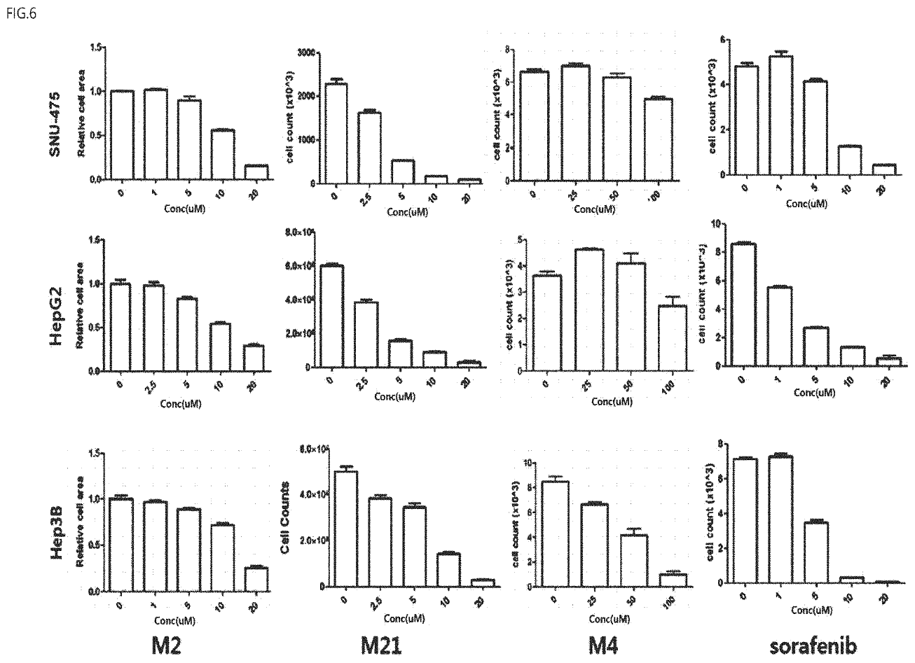

As a result, as illustrated in FIGS. 5A, 5B, 6, 8, and 9A, it was confirmed that the number of cells was remarkably reduced in proportion to the treatment concentration of the compounds.

In contrast, as illustrated in FIG. 5A, it could be seen that when a normal cell line HDF was treated with the compound, the treatment did not significantly affect apoptosis up to 20 uM.

As a result of analysis, as in FIGS. 5A to 9A, it was confirmed that the variants of the hit compound according to the present invention affected the viability of cells differently. Among them, it was confirmed that the ability of Compound 2 (M2) to inhibit cancer cells effectively and consistently appeared in relatively various cells.

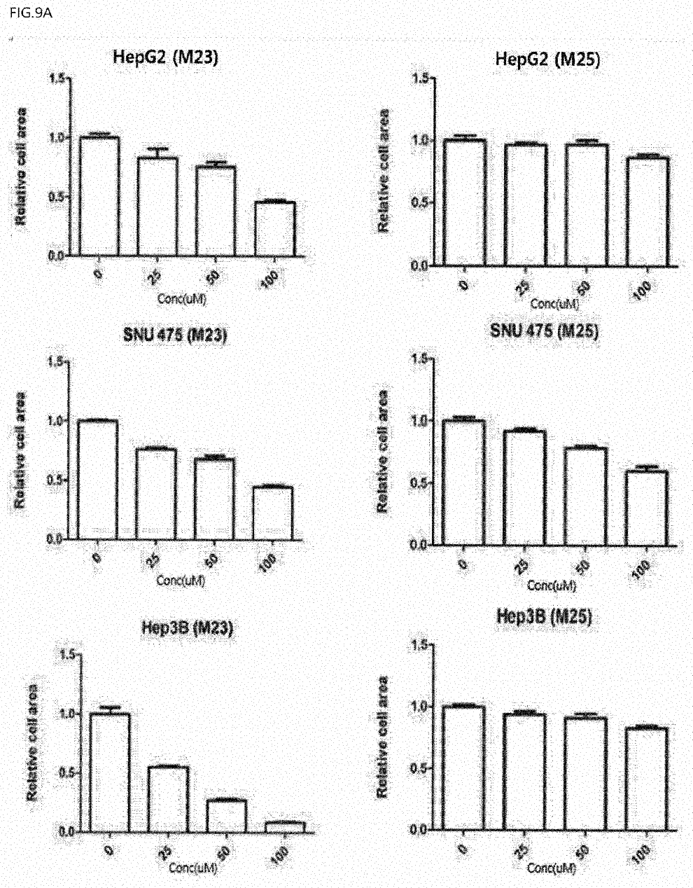

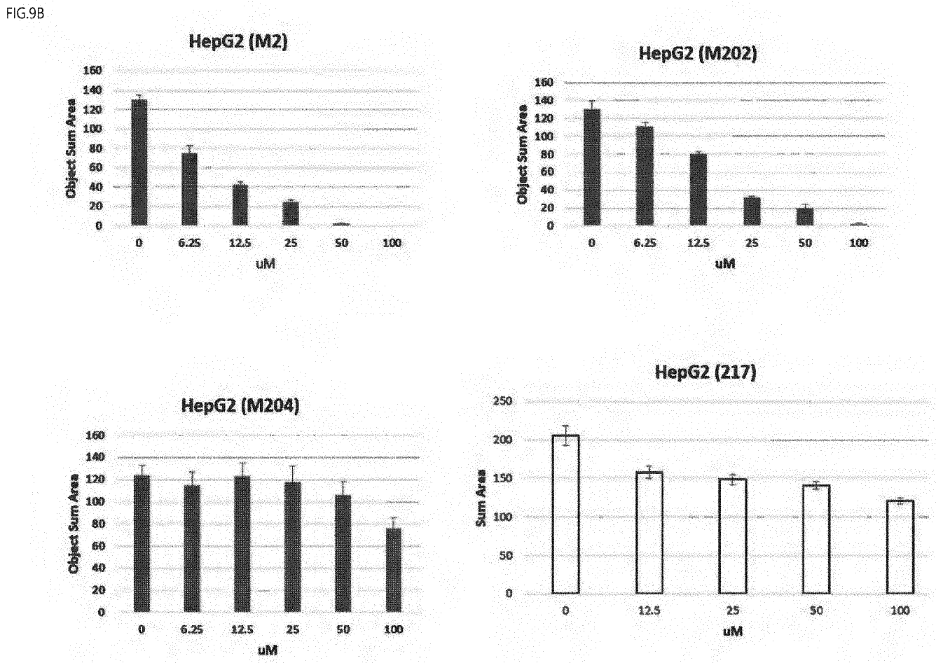

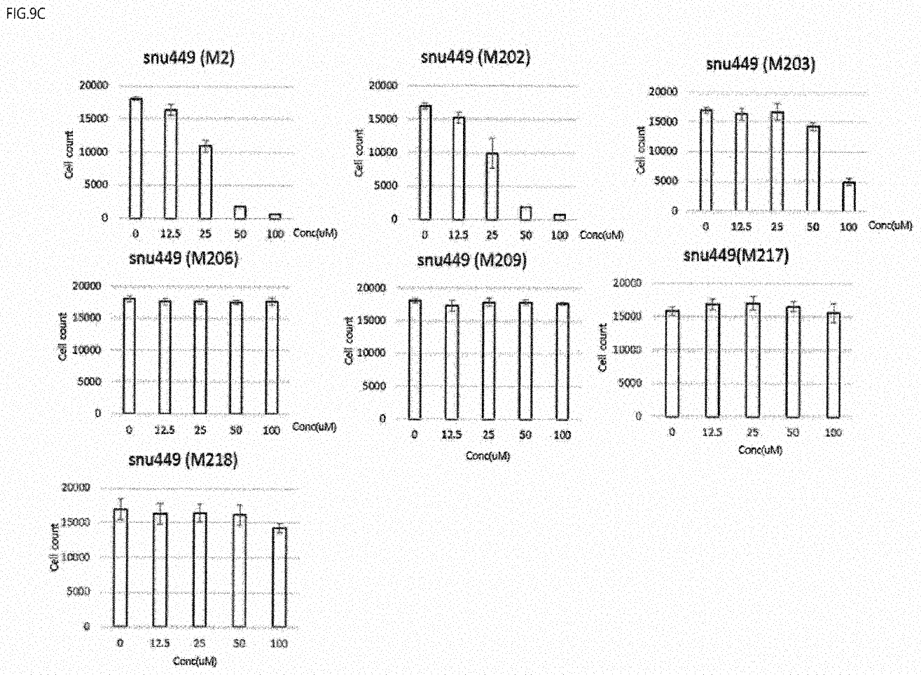

In addition, as a result of performing an experiment on the reactivity of M2 variants in HepG2 human liver cancer cells with respect to the ability to inhibit the growth of the liver cancer cell line in the same manner as in HepG2, as illustrated in FIG. 9B, the areas of liver cancer cells were remarkably reduced in a dose-dependent manner in the case of M2 and M202, but relatively low reactivity appeared in the case of M217, and as a result of performing an experiment on the reactivity of M2 variants in SNU449 human liver cancer cells, as illustrated in FIG. 9C, in the case of M2, M202, and M203, the number of cancer cells was remarkably reduced in a dose-dependent manner whereas in the case of M206, M209, M217, and M218, a relatively remarkable cancer cell reduction effect did not appear.

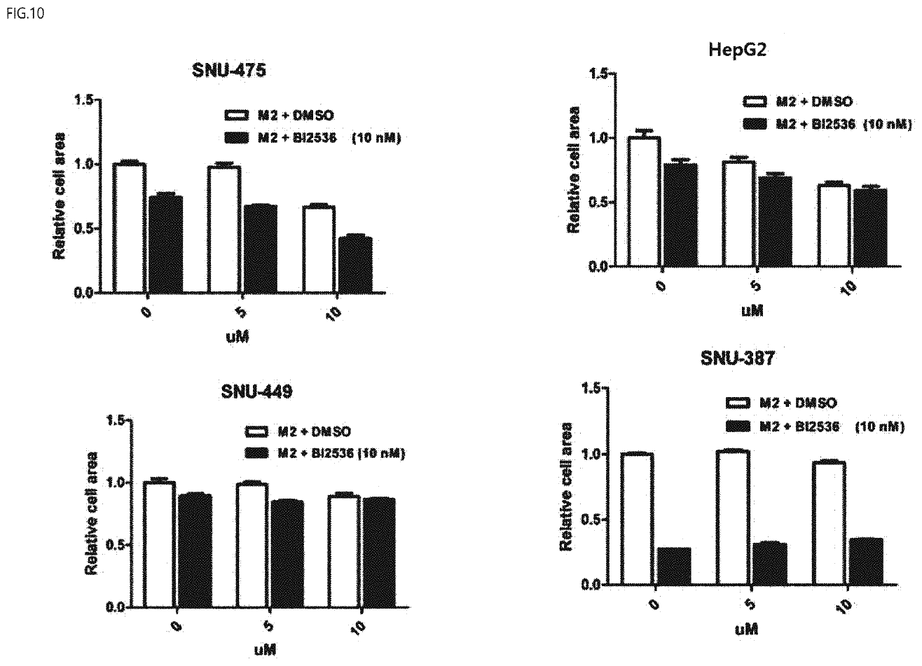

Furthermore, when cancer cells were treated with a mixture of Compound 2 (M2) at a low concentration and BI2536, which is a PLK1 kinase inhibitor, as illustrated in FIG. 10, the cooperative ability of BI2536 to inhibit cancer cells could be observed along with the reactivity of Compound 2 (M2).

### Example 4. Confirmation of Changes in PLK1 Position Due to Hit Compound and Derivative Compounds, and Comparison of Degree of Staining of NCAPG2 in Chromosome Arm and Kinetochore

In order to confirm whether there is a change in PLK1 position inside cell after cells were treated with the compound, a mutual positional relationship between r-tubulin and PLK1 located in the centrosome and the chromosome (DAPI) was confirmed.

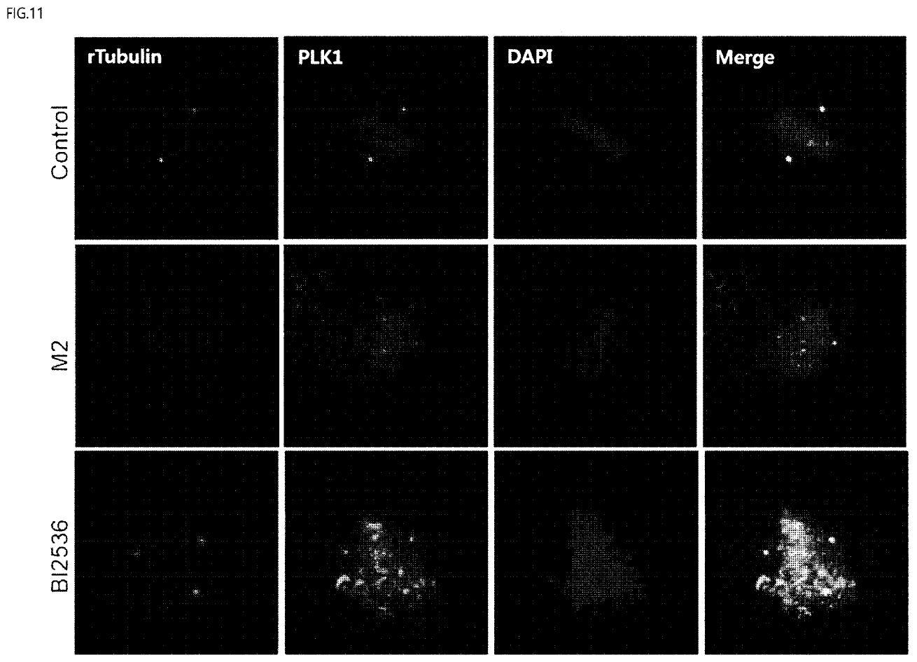

As illustrated in FIG. 11, it could be seen that PLK1 was clearly located exactly only in the central kinetochore of the centrosome and the chromosome during the middle stage of cell division (Control in FIG. 11). However, it could be seen that in the case of treatment with Compound 2 (M2), r-tubulin located in the centrosome was also weakly stained, and it also became difficult for PLK1 to be located in a normal position such that it became difficult to confirm a clear position (M2 in FIG. 11).

In contrast, treatment with BI2536 did not seem to make a relatively large difference in the positions of PLK1 or r-tubulin itself, but abnormal chromosome segregation was observed due to abnormality of the activity thereof (BI2536 in FIG. 11).

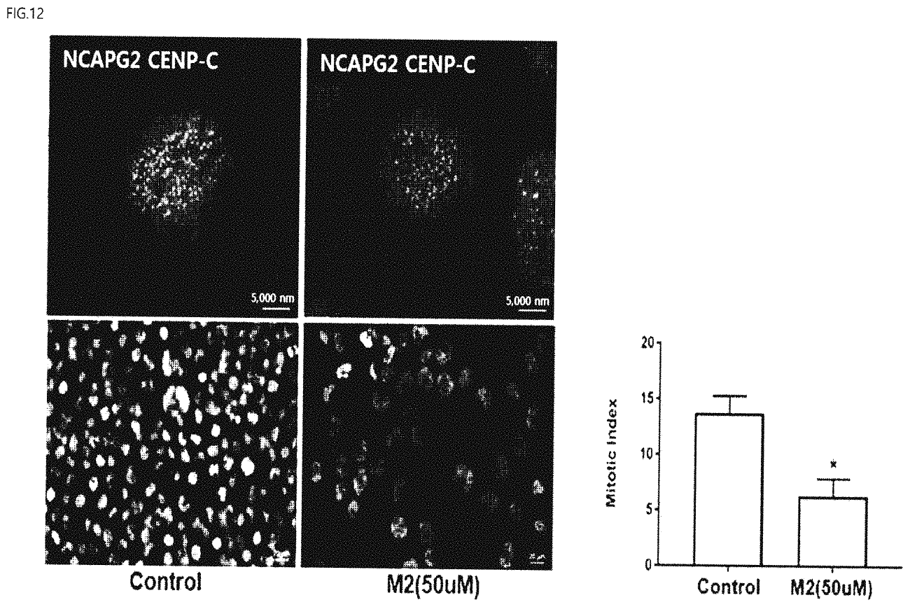

Further, as illustrated in FIG. 12, it could be confirmed that the degree of staining in the chromosome arm and kinetochore of NCAPG2, which is a PBD binding protein in the kinetochore of PLK1 was reduced in a HEK293 cell line treated with Compound 2 (M2) at a concentration of 50 uM for 24 hours (M2 in FIG. 12) compared to the control (Control in FIG. 12).

### Example 5. Confirmation of Effects of Hit Compound and Derivative Compounds on Cell Cycle

Flow cytometry equipment was used to confirm the effect on the cell cycle in the case of treatment with Compound 2 (M2). SNU-449, which is one of the liver cancer cell lines, were treated with Compound 2 (M2) at each of concentrations of 20, 40, and 80 μM after 1 day and 3 days, and were harvested after another 2 days.

Furthermore, it was intended to more specifically stain a cell population that stopped in the metaphase of cell division using a phospho-histone H3 (Ser10) antibody capable of specifically staining only the cells that stopped in the metaphase of cell division. As a positive control, cells were treated with BI2536 known as a PLK1 kinase inhibitor at 20 nM and used to observe cells in the metaphase of cell division and an increase in the G2/M phase.

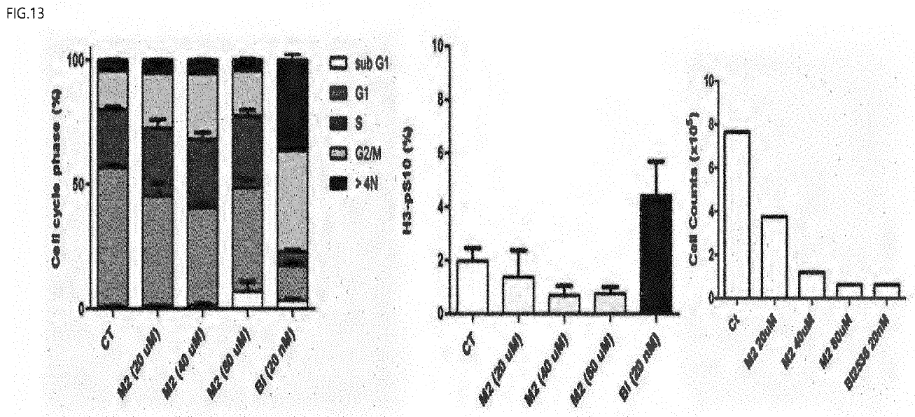

As a result of the experiment, as illustrated in FIG. 13, when cells were treated with Compound 2 (M2), the proportion of phospho-histone H3-positive cells was decreased as compared to CT, which was a result contrary to the increase in the proportion when cells were treated with B12536.

Further, as for the cell cycle, it could also be seen that when cells were treated with Compound 2 (M2), the proportion of cells having a polyploid number of chromosomes was not increased at all the treated concentrations, whereas when cells were treated with BI2536, the proportion of polyploid cells was remarkably increased (FIG. 13). Through this, it could be seen that Compound 2 (M2) affects the growth and death of cells in a different manner from BI2536, and through the fact that the proportion of polyploid cells was not increased, it could be seen that the growth of cells was suppressed before entering the cell division cycle, and the proportion of polyploid cells was not increased.

Through the results, it was confirmed that the compounds that bind to the PBD domain of PLK1 discovered through Examples 1 and 2 actually efficiently inhibited the growth of liver cancer, breast cancer, hematologic cancer, cervical cancer, and prostate cancer cells, and it could be confirmed that the inhibitory effect on the relative growth of normal cells was relatively small.

Furthermore, it was confirmed that the compounds act differently from phosphorylation activity as a PLK1 inhibitor involved in the normal cell division process in the cancer cells, and it was confirmed that a PBD targeting Hit material prevented the normal position of PLK1 itself in the cell to make the position of the exact partners thereof inappropriate, thereby exhibiting the effect of suppressing the progress of cell growth at the phases prior to the mitotic phase.

### Example 6. Confirmation of Changes in Cell Viability after Treatment with M2

HepG2 cells, a hepatocellular carcinoma cell line, were plated with 4 to 6 replications at each hit compound concentration in a 96-well microtiter plate supplemented with a culture medium. The hit compound dissolved in DMSO was added the next day according to the design of experiments, and the number of seeded cells was determined by the cell density reaching 80% on the final day of the protocol treated as a cell control. After 24 hours of cell seeding, cells were treated with hit compounds (M2 and BI2536) at various concentrations, and 48 hours after primary treatment, the medium was aspirated and then secondary treatment was performed. After 48 hours, cell nuclei were visualized by 2.5 μM Hoechst 33342 staining at 37° C. for 30 minutes, then the medium was aspirated and washed with a fresh medium. The plate was read using Cytation™ 3 (BioTek, USA), the cell viability was analyzed, and the results were presented as a relative percentage of viable cells after treatment with the hit compound compared to the control treatment.

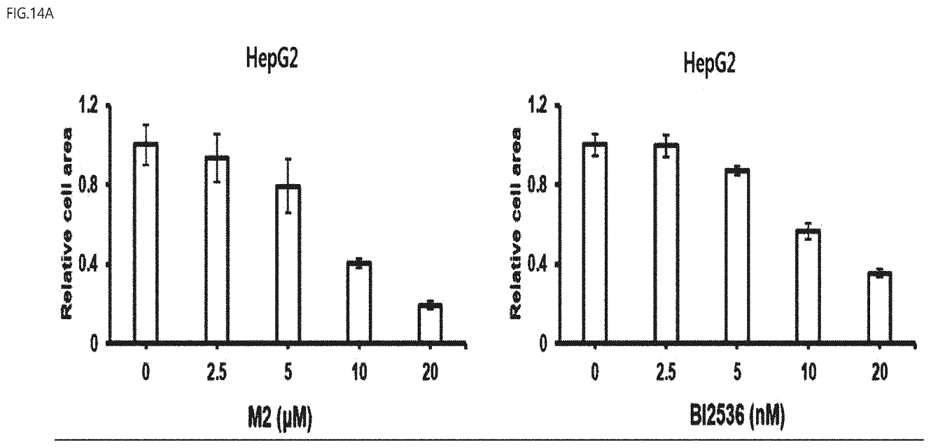

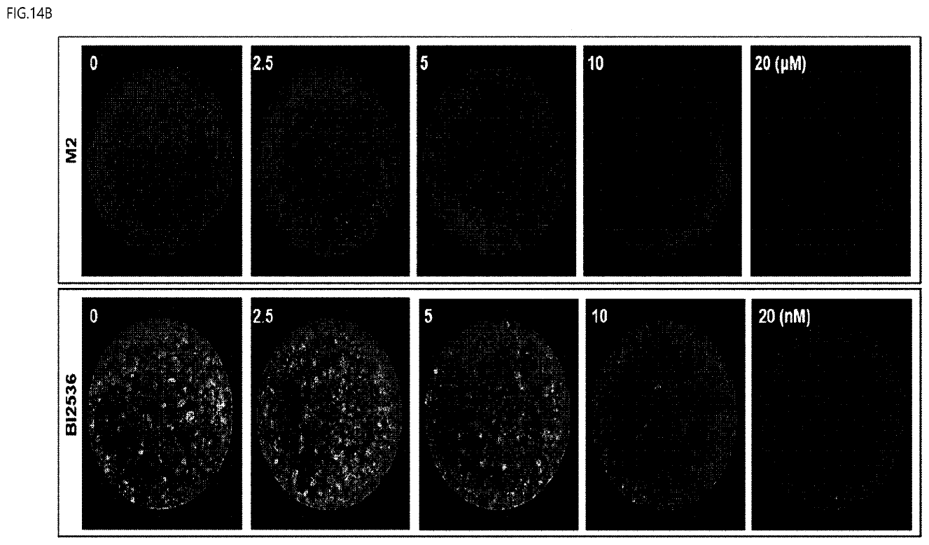

As a result, as illustrated in FIGS. 14A and 14B, it was confirmed that the higher the treated concentration of M2 and B12536 was, the relatively less the cell viability became.

### Example 7. Cell Death by Apoptosis after Treatment with M2

To analyze the pattern of apoptosis by exposure to M2, HepG2 cell, a hepatocellular carcinoma cell line, were treated with 20 or 100 μM M2 and 20 or 100 nM BI2536 for 3 days. The apoptosis was detected by annexin V-fluorescein isothiocyanate (annexin V-FITC) and propidium iodide (PI) staining of necrotic and apoptosis cells. First, cells were harvested and washed once with PBS. The cells were then resuspended in a 100 μl binding buffer containing 4 μl of Annexin V (BD, 51-65874X) and PI (BD, 51-66211E). The cells were stained at 37° C. for 15 minutes in the dark and then analyzed using FACSan (BD, San Jose, Calif.). Data was analyzed using CELLQuest software (BD).

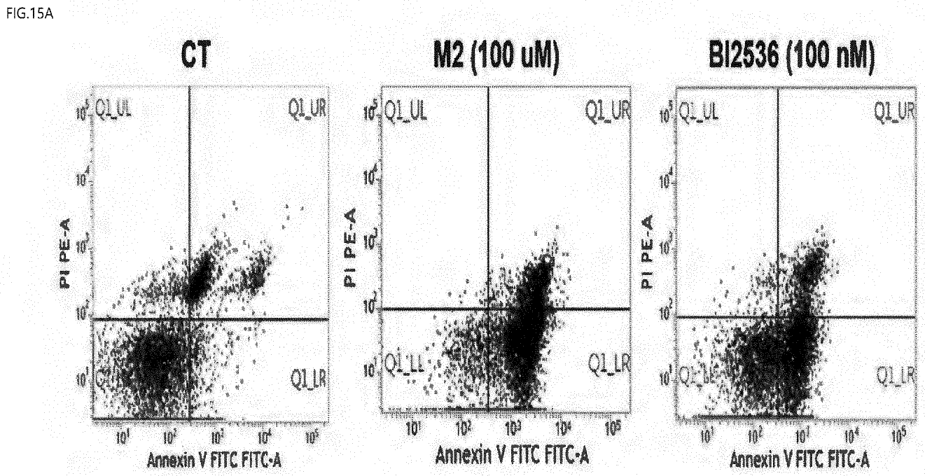

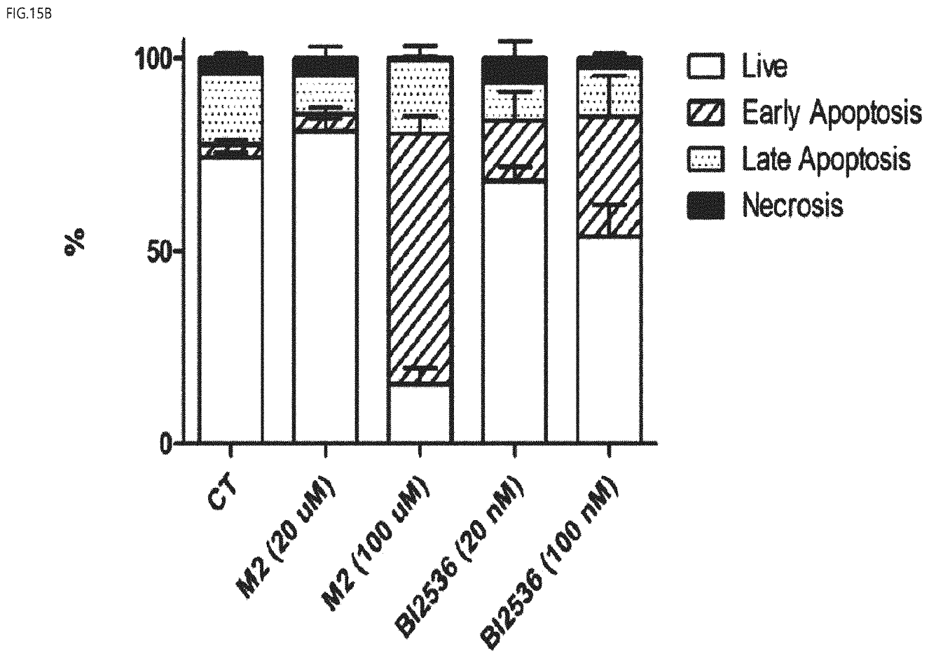

As a result, as illustrated in FIG. 15A, apoptosis cells were increased in both the M2 and BI2536 treatment groups, and as illustrated in FIG. 15B, apoptosis was increased in a dose-dependent manner in both the M2 and BI2536 treatment groups.

### Example 8. Analysis of Ability of Hit Compound Derivative Compound to Inhibit Cancer Growth in Liver Cancer Xenograft Model (Toxicity Test of Compound 4)

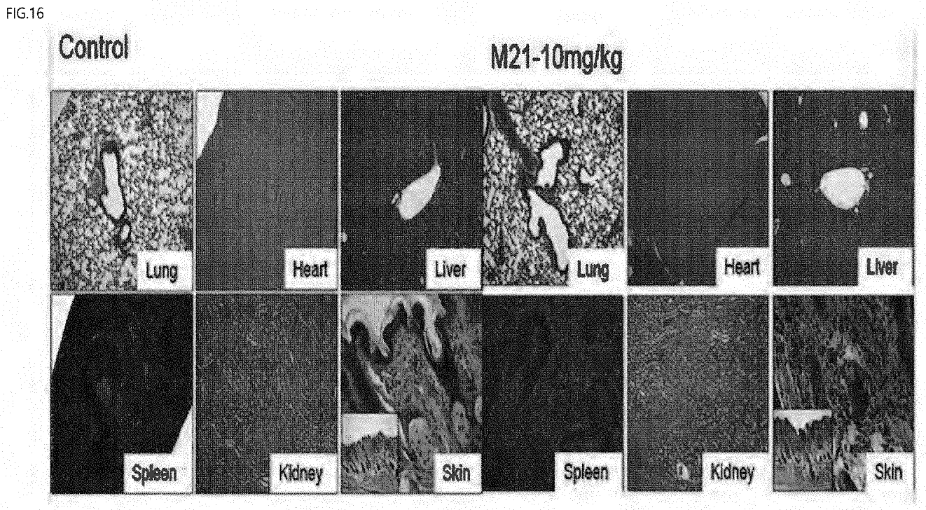

Compound 4 (M21) was diluted in 300 μl of PBS and intraperitoneally injected 3 times weekly at 1 mg/kg, 5 mg/kg and 10 mg/kg per body weight of a mouse, respectively, and DMSO diluted in 300 μl of PBS was intraperitoneally injected at 3% in the control. After 2 weeks, the lungs, heart, liver, kidneys, spleen and skin were removed by sacrificing mice, and fixed in a formalin solution. No change by separate acute toxicity was observed in a histopathological analysis of the fixed tissue (FIG. 16).

Meanwhile, a xenograft model was prepared by injecting 5×10⁶ HepG2 cell into the subcutaneous fat layer of immunodeficient mice (Balb/c-nu). After 3 weeks, each of Compound 4 (M21) and Compound 2 (M2) was diluted in 300 μl of PBS and intraperitoneally injected five times weekly at 5 mg/kg and 10 mg/kg, respectively, and DMSO diluted in 300 μl of PBS was intraperitoneally injected at 3% in the control.

Tumor size and mouse body weight were measured three times a week, and the results are illustrated in FIG. 15. Mice were sacrificed 12 days after administration of the material (administration: 10 times in total). The tumor was removed, weighed, fixed in a formalin solution, and frozen.

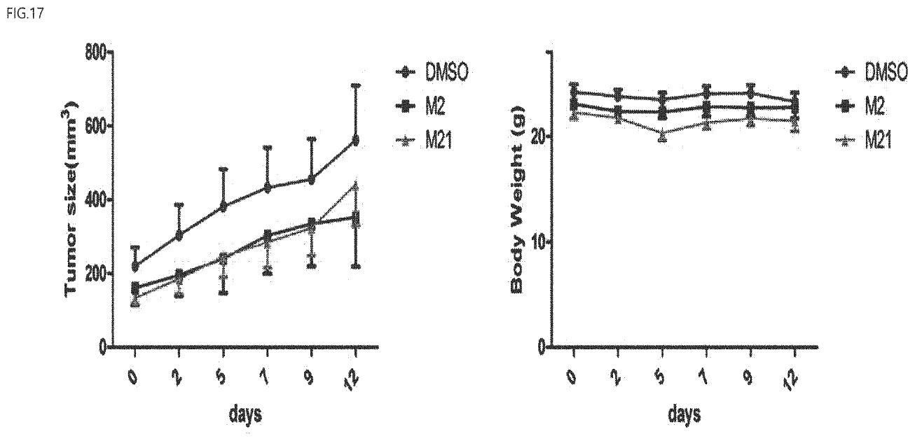

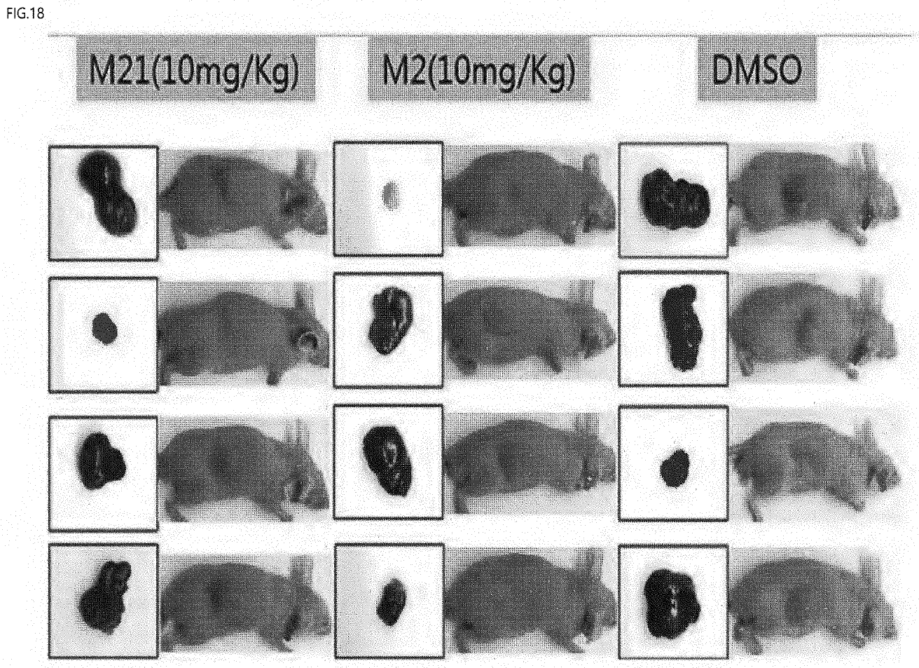

As illustrated in FIGS. 17 and 18, it could be observed that the weight of the removed cancer-producing tissue was reduced in the case of treatment with the compounds compared to the control.

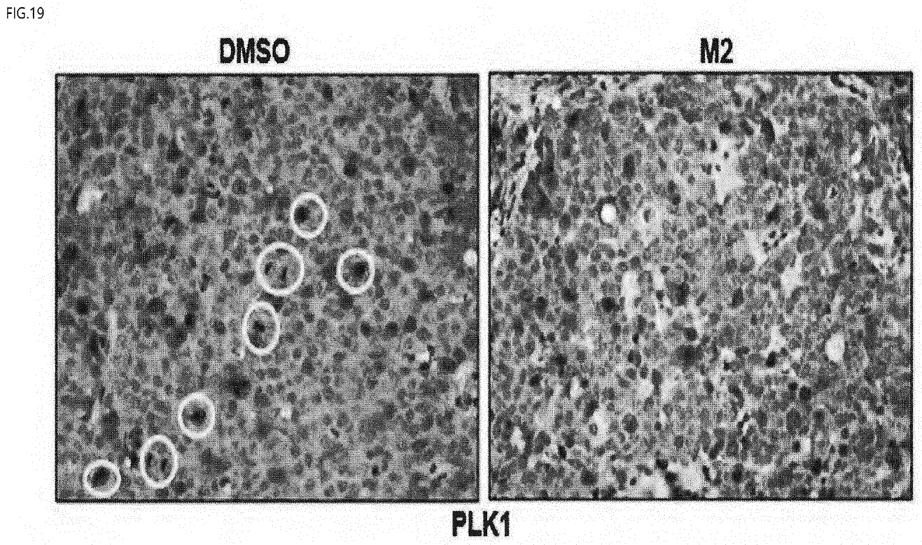

Meanwhile, as illustrated in FIG. 19, it could be seen that the difference in expression of PLK1 itself in the tissue was not remarkable, but the number of cells in the mitotic phase was reduced.

### Example 9. Analysis of Ability to Inhibit Growth of Cancer in Liver Cancer Orthotopic Xenograft Model (HepG2 Cell Line)

5×10⁶ HepG2 cells, which are a hepatocellular carcinoma cell line, were injected into the back skin of BalB/c nude mice, and when a sufficient cancer tissue was formed about 3 weeks later, the tissue was removed, uniformly cut into 1 mm³, and transplanted into the right median lobe of the liver by excising the abdomen within 1 cm.

The doses of 9.1 mg/kg of M2 and 1 mg/kg of BI2536 were selected based on the present inventor's previous in vitro experiments on HCC cells. Administration began 7 days after cell injection, and an equivalent amount of DMSO for the highest concentration of drug was used as a solvent control for each experiment. Each drug was injected a total of 19 injections based on 5 times/week.

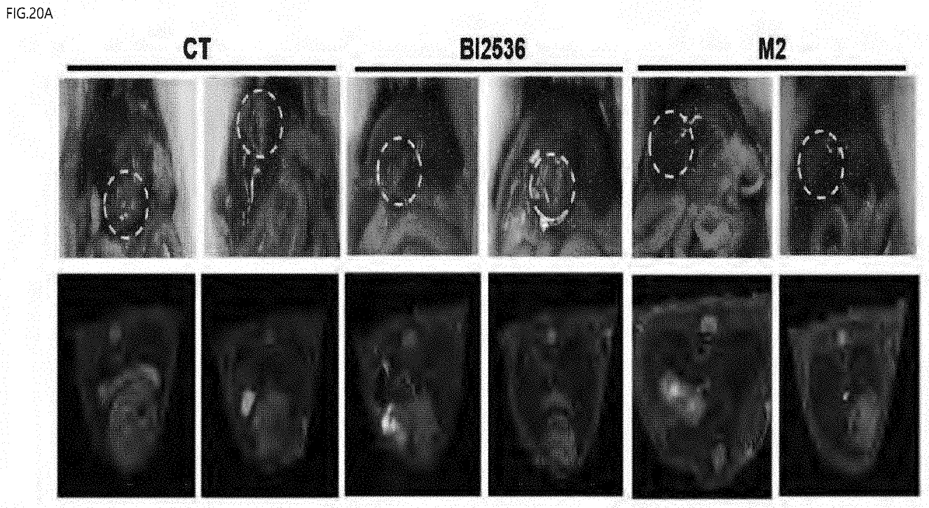

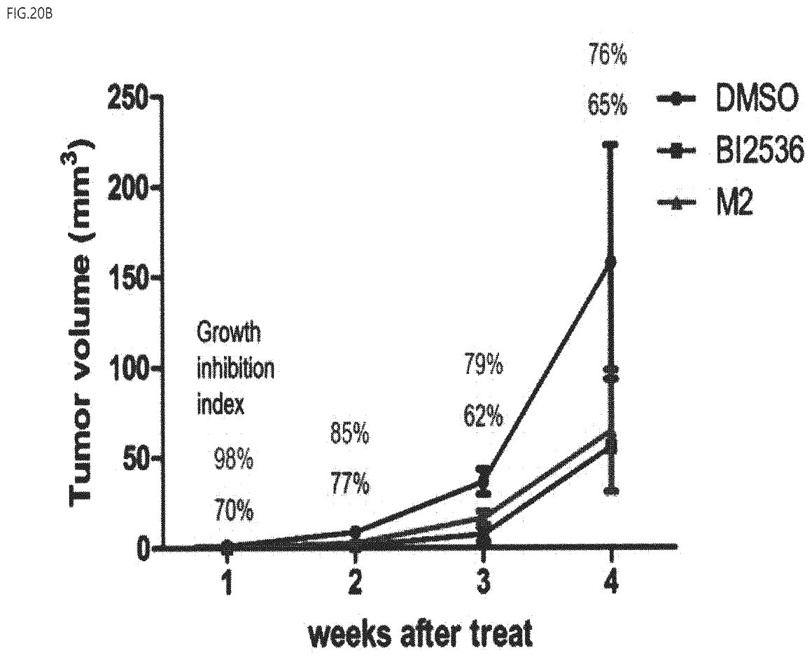

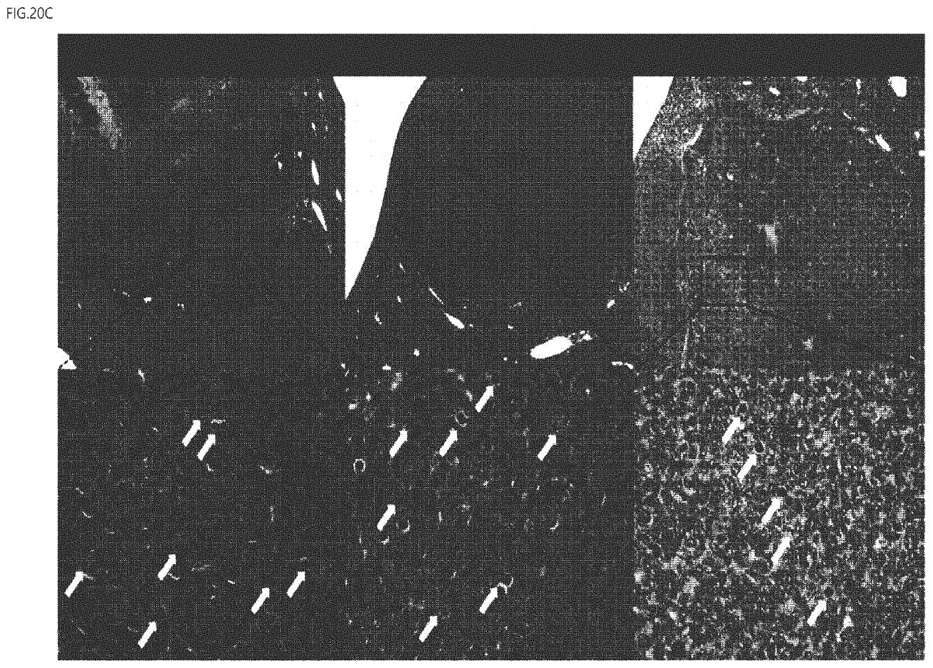

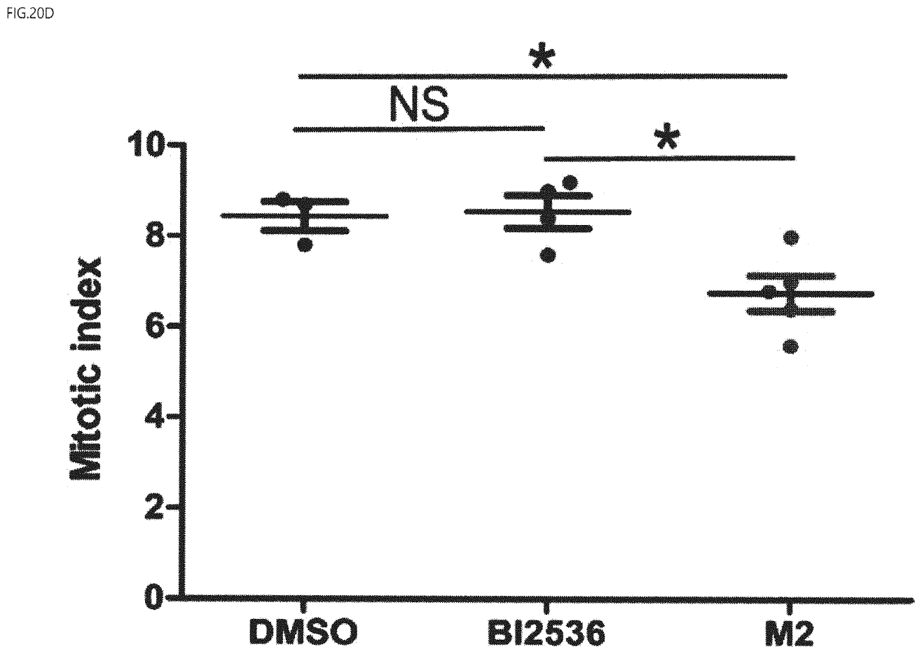

As a result, as illustrated in FIG. 20A, the growth of tumor cells was suppressed by M2 and BI2536, and as illustrated in FIG. 20B, both M2 and BI2536 suppressed the progression of the HCC xenograft calculated by the growth inhibition index. Further, it was confirmed that histological staining showed that the mitotic index was decreased in M2-treated mice compared to the control as illustrated in FIG. 20C. Decreased mitotic index in FIG. 20D was consistent with a cell cycle analysis after treatment with M2, and M2 had an action mechanism different from that of B12536 in vitro and in vivo.

Another experiment was performed on the same liver cancer cell line by varying the experimental method. 5×10⁶ HepG2 cells were injected into the backs of BalB/c nude mice, and when a sufficient cancer tissue was formed about 3 weeks later, the tissue was removed, uniformly cut into 1 mm³, and transplanted into the right median lobe of the liver by excising the abdomen within 1 cm.

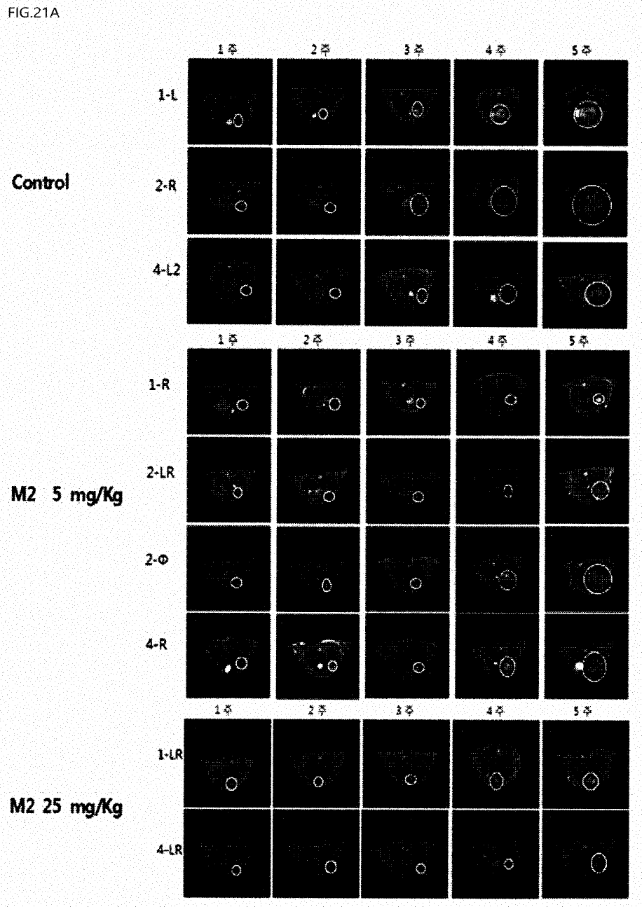

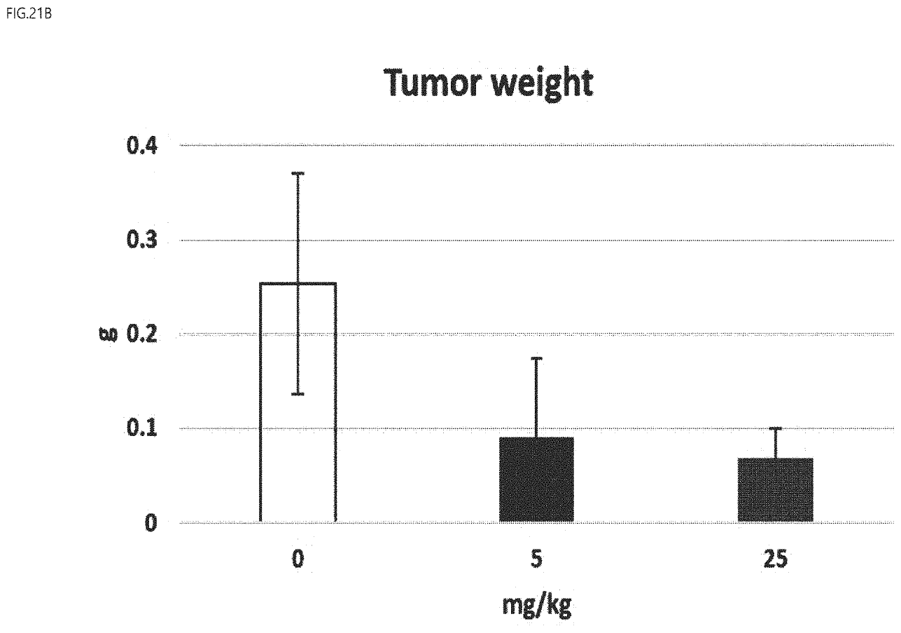

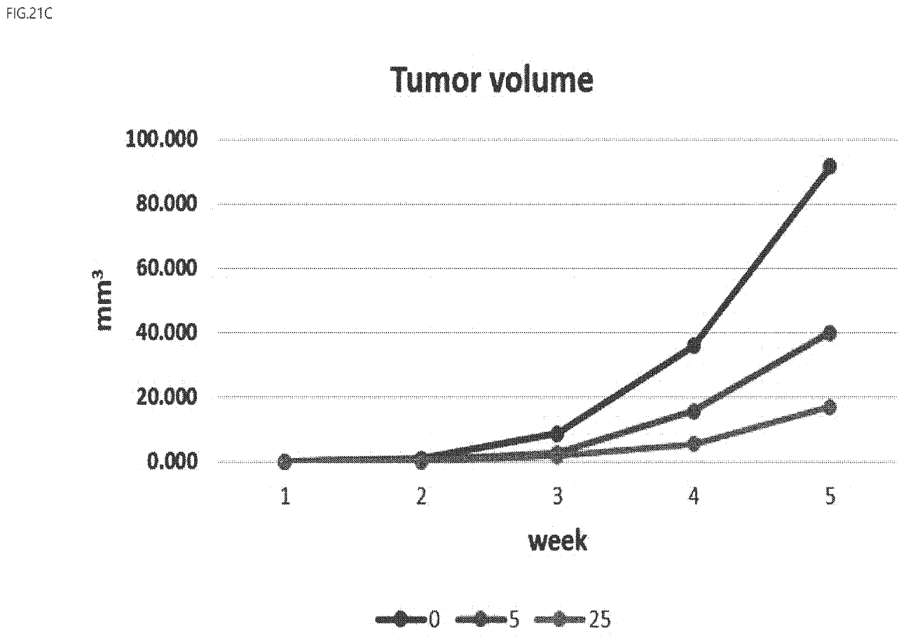

Small spots were confirmed on the MRI image about 10 days after transplanting the liver cancer tissue into 20 BalB/C nude mice by the above method, so that rodents with well-established liver cancer were divided into 3 groups (about 50 to 60%) (see FIG. 21A), a control and a hit (M2) material at 5 mg/kg and 20 mg/kg were injected into the abdominal cavity once every two days using less than 1.5% DMSO as a solvent (vehicle), and once a week, MRI images were used to select (follow up) rodents with constant tissue growth. Then, in a state in which the rapidly growing cancer tissue was 1 cm or less, the cancer tissue was observed after sacrificing the mice (10 treatments for 3 weeks).

As a result, as illustrated in FIGS. 21B and 21C, the weight and volume of the cancer tissue were reduced remarkably as the dose of M2 was increased, so that an excellent anticancer effect of M2 could be confirmed.

### Example 10. Analysis of Ability to Inhibit Growth of Cancer in Orthotopic Xenograft Model Using Human Liver Cancer PDX

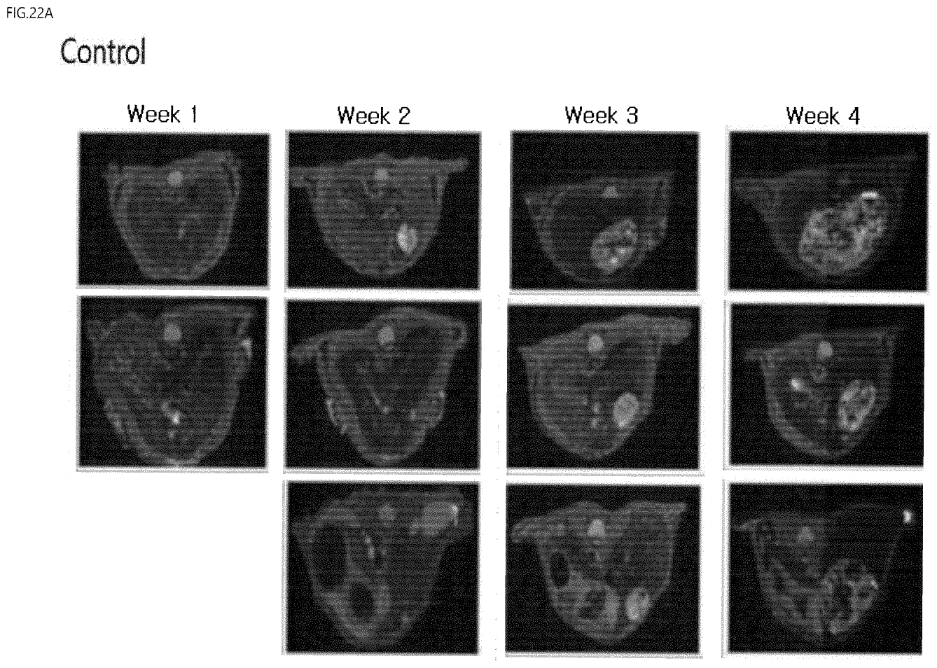

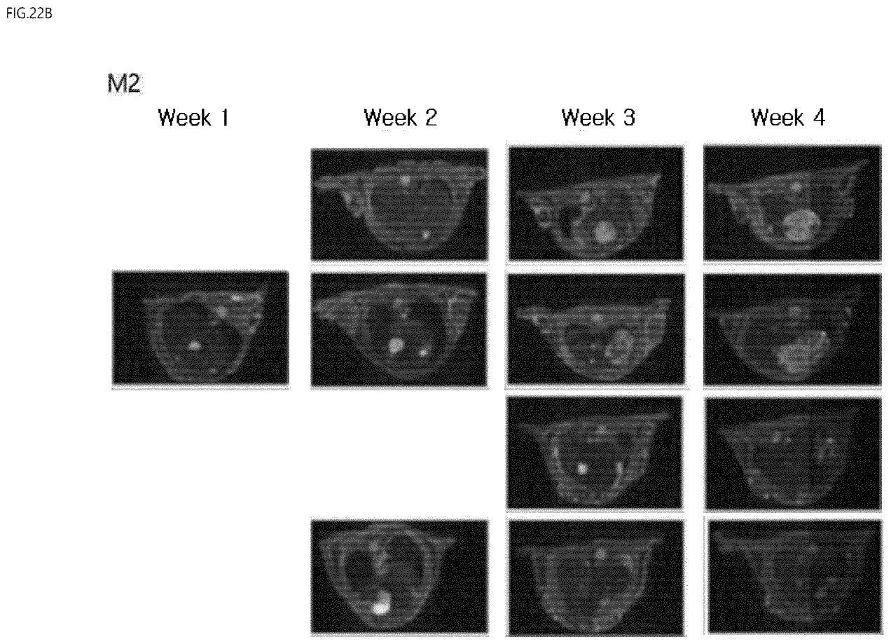

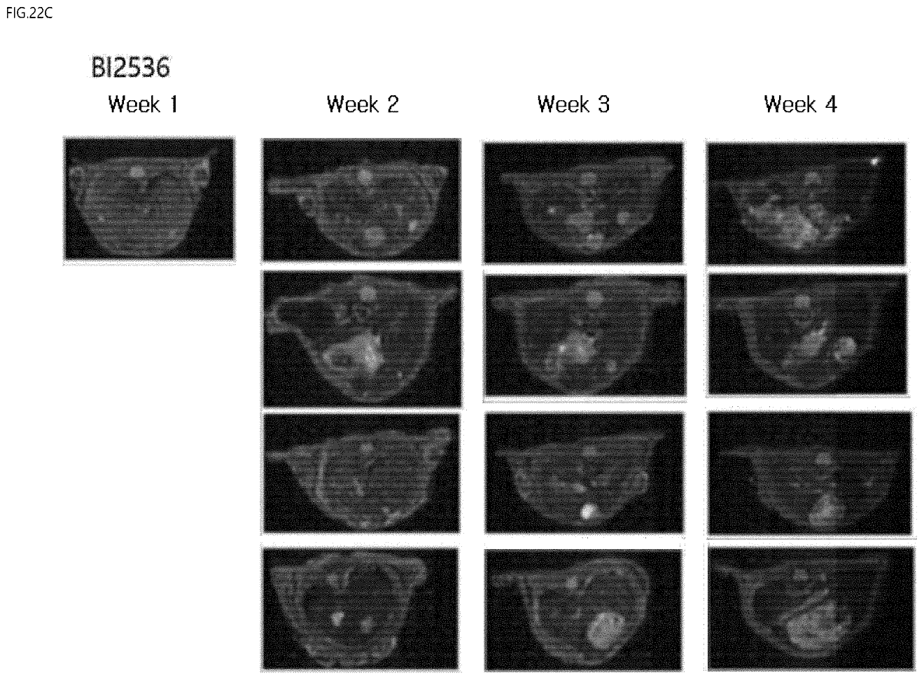

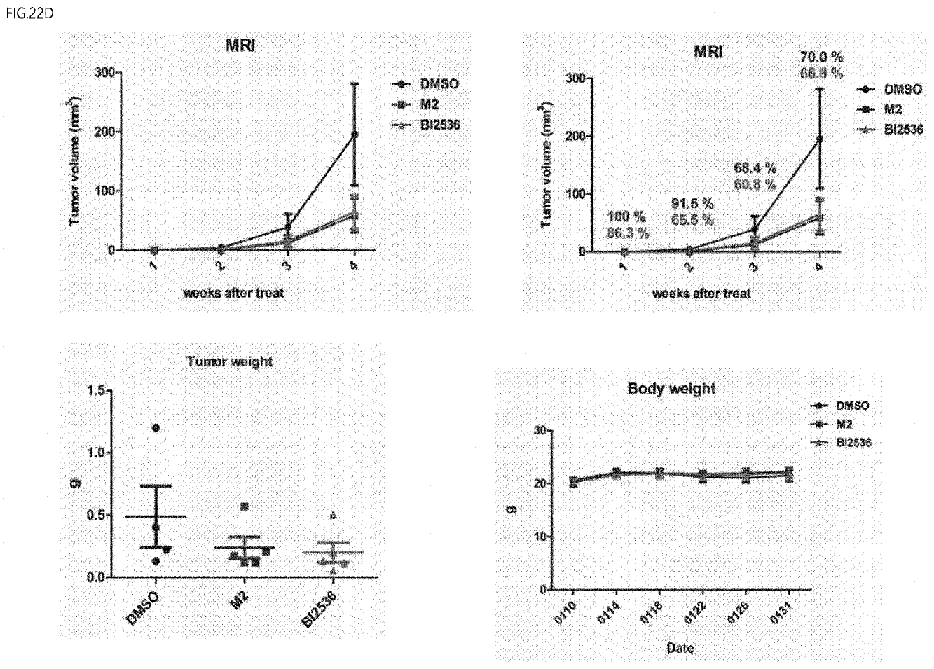

A cancer tissue isolated from human liver cancer was transplanted into skin tissues of BalB/C nude mice and the skin tissues were grown into cancer tissues, so that a tissue established as a PDX model was used, uniformly cut into 1 mm³, and transplanted into the right median lobe of the liver by excising the abdomen within 1 cm. Sm all spots were confirmed on the MRI image about 10 days after transplanting the liver cancer tissue into 20 BalB/C nude mice by the above method, so that rodents with well-established liver cancer were divided into 3 groups (see FIGS. 22A, 22B, and 22C), a solvent control (DMSO), a hit (M2) material at 40 mg/kg, and BI2536 at 4 mg/kg were injected into the abdominal cavity once every two days using less than 1.5% DMSO as a solvent (vehicle), and once a week, MRI images were used to select (follow up) rodents with constant tissue growth. Then, when the rapidly growing cancer tissue was 1 cm or less, the cancer tissue was observed after sacrificing the mice (11 treatments for 3 weeks).

As a result, as illustrated in FIGS. 22B to 22D, the weight and volume of the cancer tissue were reduced remarkably according to the administration of M2, so that an excellent anticancer effect of M2 could be confirmed.

The above-described description of the present invention is provided for illustrative purposes, and a person skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

The compounds of the present invention have advantages of having high selectivity and binding affinity and low toxicity by selectively binding to the PBD of PLK1 compared to ATP binding site inhibitors targeting a kinase domain in the related art. Therefore, the compounds of the present invention can be usefully used as an anticancer agent that inhibits the growth of various cancer cells, and a synergistic effect can be expected by co-administration with other existing anticancer agents in addition to single administration, so that the compounds can be widely used not only in the pharmaceutical industry but also in the health functional food industry.

1. A method of treating cancer, comprising:
administering a compound represented by the following Chemical Formula 1 or 2, or a pharmaceutically acceptable salt thereof, into an individual. 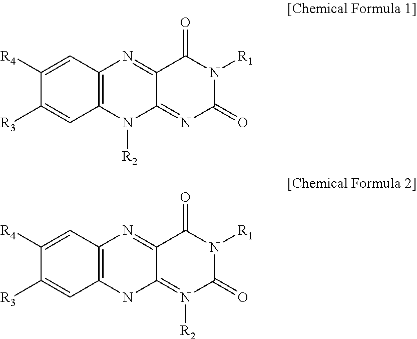
wherein in Chemical Formula 1 or 2,
R₁ is H, an alkyl, or —CₙH₂ₙCOOH, where n is an integer from 1 to 4,
R₂ is H, an alkyl, —CₘH₂ₘCN, —CₘH₂ₘOR₅, —CₚH₂ₚ(CH(OH))_{q}R₆, R₅ is a phenyl substituted with one or more C₁₋₃ alkyls, R₆ is H, an alkyl, or —OPH₂O₃, m is an integer from 2 to 4, p is an integer from 1 to 3, and q is an integer from 2 to 4,
R₃ is H, a halogen, —NH₂, an alkyl, or —CH═O, and
R₄ H, an alkyl, —COOH, or —CX₃, and X is a halogen.

2. The method of claim 1, wherein in Chemical Formula 1 or 2,
R₁ is H, —CH₃, or —CH₂COOH,
is H, —CH₃, —C₂H₄CN, —CH₂(CH(OH))₃CH₂OH, —CH₂(CH(OH))₃OPH₂O₃, or 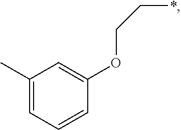
R₃ is H, Cl, —NH₂, —CH₃, or —CH═O, and
R₄ is H, —CH₃, —COOH, or —CF₃.

3. The method of claim 1, wherein the compound represented by Chemical Formula 1 or 2 is selected from the group consisting of the allowing compounds:
2,4-Dioxo-1,2,3,4-tetrahydrobenzo[g]pteridine-7-carboxylic acid;
10-methyl-2H,3H,4H,10H-benzol[g]pteridine-2,4-dione;
8-chloro-1H,2H,3H,4H-benzo[g]pteridine-2,4-dione;
10-methyl-7-(trifluoromethyl)-2H,3H,4H,10H-benzo[g]pteridine-2 diode;
8-amino-1,3-dimethyl-1H,2H,3H,4H-benzo[g]pteridine-2,4-dione;
8-amino-2H,3H,4H,10H-benzo[g]pteridine-2,4-dione;
7,8,10-trimethyl-2H,3H,4H,10H-benzo[g]pteridine-2,4-dione;
7,10-dimethyl-2,4-dioxo-2H,3H,4H,10H-benzo[g]pteridine-8-carbaldehyde;
4,10-Dihydro-7,8,10-trimethyl-2,4-dioxobenzo[g]pteridine-3(2H)-acetic acid;
3-{7,8-dimethyl-2,4-dioxo-2H,3H,4H,10H-benzo[g]pteridin-10-yl}propanenitrile;
10-[2-(3-methylphenoxy)ethyl]-7-(trifluoromethyl)-2H,3H,4H,10H-benzo[g]pteridine-2,4-dione;
7,8-dimethyl-10-[(2S,3S,4R)-2,3,4,5-tetrahydroxypentyl]benzo[g]pteridine-2,4-dione; and
[(2R,3S,4S)-5-(7,8-dimethyl-2,4-dioxobenzo[g]pteridin-10-yl)-2,3,4-trihydroxypentyl] dihydrogen phosphate.

4. The method of claim 1, wherein the cancer is one or more selected from the group consisting of liver cancer, breast cancer, hematologic cancer, cervical cancer, and prostate cancer.

5. The method of claim 1, wherein the compound binds to a polo-box do s domain (PBD) of polo-like kinase 1 (PLK1).

6. The method of claim 1, wherein the compound represented by Chemical Formula 1 or 2, or the pharmaceutically acceptable salt thereof inhibits the growth of cancer cells.

7. The pharmaceutical composition method of claim 1, wherein the compound represented by Chemical Formula 1 or 2, or the pharmaceutically acceptable salt thereof induces apoptosis of cancer cells.

8. A health functional food composition for alleviating cancer, comprising a compound represented by the following Chemical Formula 1 or 2, or a pharmaceutically acceptable salt thereof as an active ingredient. 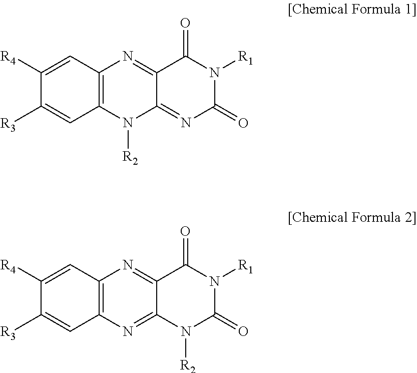
in Chemical Formula 1 or 2,
R₁ is H, an alkyl, or —CFₙH₂ₙCOOH, where n is an integer from 1 to 4,
R₂ is H, an alkyl, —CₘH₂ₘCN, —CₘH₂ₘOR₅, or —CₚH₂ₚ(CH(OH))_{q}R₆, R₅ is a phenyl substituted with one or mote C₁₋₃ alkyls, R₆ is H, an alkyl, or —OPH₂O₃, m is an integer from 2 to 4, p is an integer from 1 to 3, and q is an integer from 2 to 4,
R₃ is H, a halogen, —NH₂, an alkyl, or —CH═O, and
R₄ is H, an alkyl, —COOH, or —CX₃, and X is a halogen.

9. The health functional food composition of claim 8, wherein in Chemical Formula 1 or 2,
R₁ is H, or —CH₂COOH,
R₁, is H, —C₂H₄CN, —CH₂(CH(OH))₃CH₂OH, —CH₂(CH(OH)₃OPH₂O₃, or 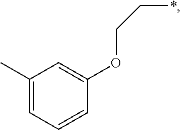
R₃ is H, —NH₂, —CH₃ or —CH═O, and
R₄ is H, —CH₃, —COOH, or —CF₃.

10. The health functional food composition of claim 8, wherein the compound represented by Chemical Forma or 2 is selected from the group consisting of the following, compounds:
2,4-Dioxo-1,2,3,4-tetrahydrobenzo[g]pteridine-7-carboxylic acid;
10-methyl-2H,3H,4H,10H-benzo[g]pteridine-2,4-dione;
8-chloro-1H,2H,3H,4H-benzo[g]pteridine-2,4-dione;
10-methyl-7-(trifluoromethyl)-2H,3H,4H,10H-benzo[g]pteridine-2,4-dione;
8-amino-1,3-dimethyl-1H,2H,3H,4H-benzo[g]pteridine-2,4-dione;
8-amino-2H,3H,4H,10H-benzo[g]pteridine-2,4-dione;
7,8,10-trimethyl-2H,3H,4H,10H-benzo[g]pteridine-2,4-dione;
7,10-dimethyl-2,4-dioxo-2H,3H,4H,10H-benzo[g]pteridine-8-carbaldehyde;
4,10-dihydro-7,8,10-tri methyl-2,4-di oxobenzo[g]pteridine-3 (2H)-acetic acid;
3-{7,8-dimethyl-2,4-dioxo-2H,3H,4H,10H-benzo[g]pteridin-10-yl}propanenitrile;
10-[2-(3-methylphenoxy)ethyl]-7-(trifluoromethyl)-2H,3H,4H,10H-benzo[g]pteridine-2,4-dione;
7,8-dimethyl-10-[(2S,3S,4R)-2,3,4,5-tetrahydroxypentyl]benzo[g] pteridine-2,4-dione; and
[(2R,3S,4S)-5-(7,8-dimethyl-2,4-dioxobenzo[g]pteridin-10-yl)-2,3,4-trihydroxypentyl] dihydrogen phosphate.

11-12. (canceled)
